(19) United States
(12) Patent Application Publication
(10) Pub. No.: US 2012/0083421 A1
(43) Pub. Date: Apr. 5, 2012

(54) USE OF URINARY NGAL TO DIAGNOSE AND MONITOR HIV-ASSOCIATED NEPHROPATHY (HIVAN)

(75) Inventors: Jonathan Barasch, New York, NY (US); Neal Paragas, New York, NY (US)

(73) Assignee: THE TRUSTEES OF COLUMBIA UNIVERSITY IN THE CITY OF NEW YORK, New York, NY (US)

(21) Appl. No.: 13/087,987

(22) Filed: Apr. 15, 2011

Related U.S. Application Data
(63) Continuation-in-part of application No. PCT/US09/61050, filed on Oct. 16, 2009.
(60) Provisional application No. 61/106,095, filed on Oct. 16, 2008.
(60) Provisional application No. 61/441,435, filed on Feb. 10, 2011.

(51) Int. Cl. C12Q 1/70; C40B 30/04

(52) U.S. Cl. 506/9; 435/5

(57) ABSTRACT

The present invention is directed to methods for diagnosis of HIVAN, and to methods for distinguishing between HIVAN and other kidney diseases, based on the presence in a bodily fluid, such as urine, of a level of NGAL protein that exceeds a threshold level. The present invention is also directed to methods for diagnosis of HIVAN based on the differential expression of NGAL and one or more other markers, such as uL-FABP or KIM-1, and to methods of diagnosis based on a specific localization of NGAL in microcysts or microcystic tubules of the kidney.

This application is a continuation-in-part of International Patent Application PCT/US2009/61050, filed Oct. 16, 2009, which claims the benefit of the filing date of U.S. Provisional Patent Application No. 61/106,095, filed Oct. 16, 2008, and this application also claims the benefit of the filing date of U.S. Provisional Patent Application No. 61/441,435, filed Feb. 10, 2011, the contents of each of which are hereby incorporated by reference in their entireties.

All patents, patent applications and publications cited herein are hereby incorporated by reference in their entirety.

### GOVERNMENT INTERESTS

This invention was made with government support under grant number RO1 DK07346202 awarded by the National Institutes of Health and grant numbers DK-55388 and DK-58872 awarded by National Institute of Diabetes and Digestive and Kidney Diseases. Accordingly, the United States Government has certain rights to the invention.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

An important complication of HIV is a form of kidney disease called HIV-associated nephropathy (HIVAN), which occurs predominantly in patients of African descent. Rao et al., N. Engl. J. Med. 310: 669-673, (1984); Winston et al., Kidney Int. 55: 1036-1040, (1999); Lucas et al., AIDS 18: 541-546, (2004). The prevalence of HIVAN may be as high as 15% of HIV patients (based upon autopsy data), and 4232 new cases of HIVAN reached end-stage renal disease (ESRD) between 2002 and 2006 in the United States. Shahinian et al., Am. J. Kidney Dis. 35: 884-888, (2000).

HIVAN is a rapidly progressive form of chronic kidney disease (CKD) characterized by nephrotic range proteinuria. Kidney biopsies demonstrate histologic abnormalities in both glomeruli and tubules, including collapsing focal segmental glomerulosclerosis (FSGS), podocyte proliferation and dedifferentiation, tubular dilation, microcyst formation, and tubulointerstitial inflammation. Atta et al., Expert Rev. Anti. Infect. Ther. 6: 365-371, (2008); Wyatt et al., Semin. Nephrol. 28: 513-522, (2008). The pathogenesis is believed to be due to dysregulation of podocytes and tubular epithelia by HIV-1 itself. Herman et al., Semin. Nephrol. 23: 200-208, (2003); Bruggeman et al., J. Clin. Invest. 100: 84-92, (1997). Early identification of HIVAN is important because highly active antiretroviral therapy (HAART), corticosteroids, and inhibition of renin-angiotensin may delay disease progression. Atta et al, Expert Rev. Anti. Infect. Ther. 6: 365-371, (2008); Post et al., Clin. Infect. Dis. 46: 1282-1289, (2008); Gupta et al., Clip. Infect. Dis. 40: 1559-1585, (2005). Nonetheless, because HIV infection may be associated with other glomerular disease, definitive diagnosis of HIVAN requires a kidney biopsy. In fact, half of all patients with presumed HIVAN demonstrated different types of lesions once biopsied. Gupta et al., Clin. Infect. Dis. 40: 1559-1585, (2005); Szczech et al., Kidney Int. 66: 1145-1152, (2004).

Neutrophil gelatinase-associated lipocalin (NGAL) is a 22-kD protein that is markedly upregulated in renal tubules and urine (uNGAL) in response to epithelial damage. Nickolas et al., Ann. Intern. Med. 148: 810-819, (2008); Mori et al., J. Clin. Invest. 115: 610-621, (2005); Mishra et al. J. Am. Soc. Nephrol. 14: 2534-2543, (2003). Expression of NGAL peaks 12 h after acute injury (Mori et al., (2005); Mishra et al., (2003)), but remains elevated if injury is severe (Schmidt-Ott et al., J. Am. Soc. Nephrol. 18: 407-413, (2007)).

### SUMMARY OF THE INVENTION

The present invention is based, in part, on the surprising discovery that levels of Neutrophil Gelatinase-Associated Lipocalin (NGAL) protein in the urine of patients with HIV associated nephropathy (HIVAN) are much higher than the levels of NGAL in the urine of both HIV-positive and HIV-negative patients with no kidney disease or even with other forms of chronic kidney disease. The levels of NGAL protein in the urine of patients with HIVAN are much higher than could have been predicted based on the extent of the kidney disease and the level of impairment of kidney function.

In one embodiment, the present invention provides a method for determining whether a subject has HIV associated nephropathy (HIVAN), the method comprising measuring the amount of NGAL protein in the urine of the subject, wherein an amount of NGAL protein in the urine that exceeds about 120 μg/g creatinine indicates that the subject has HIVAN. In one aspect, the subject is of African descent, and/or has already been determined to be HIV positive. In another aspect, the subject has no other signs of kidney disease at the time that the method is performed, and the method is used for the early detection of HIVAN before the onset of other symptoms.

In another embodiment, the present invention provides a method for distinguishing between HIV associated nephropathy (HIVAN) and non-HIVAN kidney disease in a subject, the method comprising measuring the amount of NGAL protein in the urine of the subject, wherein an amount of NGAL protein in the urine that exceeds about 100 μg/g creatinine indicates that the subject has HIVAN and an amount of NGAL protein in the urine that is less than about 120 μg/g creatinine indicates that the subject has a non-HIVAN kidney disease. In one aspect, the subject is of African descent, and/or has already been determined to be HIV positive. In another aspect, the subject has no other signs of kidney disease at the time that the method is performed, and the method is used for the early detection of HIVAN before the onset of other symptoms.

The above methods can also be used to determine if the subject has HIV-associated microcystic degeneration of the kidney or collapsing focal segmental glomerulosclerosis (collapsing FSGS).

In some embodiments, the methods of the invention further comprise measuring the amount of KIM-1 protein in the urine of the subject, wherein an amount of NGAL protein that is more than about 8-fold higher than the amount of KIM-1 protein, in combination with an amount of NGAL protein that exceeds about 100 μg/g creatinine, indicates that the subject has HIVAN.

In other embodiments, the methods of the invention further comprise determining whether the subject has proteinuria, wherein proteinuria, in combination with an amount of NGAL protein in the urine that exceeds about 120 μg/g creatinine, indicates that the subject has HIVAN.

In further embodiments, the methods of the invention further comprise determining the subject's CD4 count, wherein a CD4 count of less than about 200 cells/mm³, in combination with an amount of NGAL protein in the urine that exceeds about 100 μg/g creatinine, indicates that the subject has HIVAN.

In other embodiments, the methods of the invention further comprise determining the subject's serum creatinine level, wherein a serum creatinine level of more than about 1.5 mg/dl, in combination with an amount of NGAL protein in the urine that exceeds about 100 μg/g creatinine, indicates that the subject has HIVAN.

In yet other embodiments, the methods of the invention further comprise determining the subject's HIV viral load, wherein an HIV viral load of more than about 100,000 IU/ml, in combination with an amount of NGAL protein in the urine that exceeds about 100 μg/g creatinine, indicates that the subject has HIVAN.

In other embodiments, the methods of the invention further comprise determining the localization of NGAL mRNA or NGAL protein in the kidney of the subject, wherein the localization of NGAL mRNA or NGAL protein in microcysts, in combination with an amount of NGAL protein in the urine that exceeds about 100 μg/g creatinine, indicates that the subject has HIVAN. According to such a method, the localization of NGAL mRNA or NGAL protein is preferably determined using a kidney biopsy sample from the subject.

In another embodiment, the methods of the invention further comprise determining the localization of NGAL mRNA or NGAL protein in the kidney of the subject, wherein presence of NGAL mRNA or NGAL protein in microcystic tubules but not in non-cystic tubules, in combination with an amount of NGAL protein in the urine that exceeds about 120 μg/g creatinine, indicates that the subject has HIVAN. According to such a method, the localization of NGAL mRNA or NGAL protein is preferably determined using a kidney biopsy sample from the subject.

In some embodiments, the methods of the invention comprise measuring the amount of uL-FABP protein in the urine of the subject, wherein an amount of uL-FABP that exceeds about 115 μg/g creatinine, indicates that the subject has HIVAN.

In some embodiments, the methods of the invention comprise measuring the amount of uL-FABP protein in the urine of the subject and measuring the amount of uNGAL in the urine of the subject, wherein an amount of uL-FABP that exceeds about 115 μg/g creatinine, in combination with an amount of NGAL protein that exceeds about 120 μg/g creatinine, indicates that the subject has HIVAN.

In a further embodiment, the present invention provides a method for determining whether a human subject is a candidate for treatment with highly active antiretroviral therapy (HAART), the method comprising obtaining a urine sample from the subject; and measuring the amount of NGAL protein in the urine sample, wherein an amount of NGAL protein in the urine that exceeds about 120 μg/g creatinine indicates that the subject is a candidate for treatment with highly active antiretroviral therapy (HAART). In some embodiments, such methods further comprise a subsequent step of administering highly active antiretroviral therapy (HAART) to the subject.

In other embodiments, the present invention provides a method for monitoring the progression of HIVAN in a subject, the method comprising: a) obtaining a first urine sample from the subject at a first time point; (b) measuring the amount of NGAL protein in the first urine sample, (c) obtaining a second urine sample from the subject at a second time point that is after the first time point; and (d) measuring the amount of NGAL protein in the second urine sample, wherein an amount of NGAL protein in the first urine sample that exceeds about 120 μg/g creatinine indicates that the subject has HIVAN, and wherein an amount of NGAL protein in the second urine sample that exceeds the amount of NGAL protein in the first urine sample, indicates that the HIVAN is worsening, and an amount of NGAL protein in the second urine sample that is less than the amount of NGAL protein in the first urine sample, indicates that the HIVAN is improving. In certain embodiments of such methods the first urine sample is obtained before the initiation of HAART therapy in the subject and the second urine sample is obtained after the initiation of HAART therapy in the subject, such that an amount of NGAL protein in the second urine sample that exceeds the amount of NGAL protein in the first urine sample, indicates that the HAART has not been effective, and an amount of NGAL protein in the second urine sample that is less than the amount of NGAL protein in the first urine sample, indicates that the HAART has been effective. In other embodiments of such methods the first and second urine sample can both be obtained after the initiation of HAART therapy, such that an amount of NGAL protein in the second urine sample that exceeds the amount of NGAL protein in the first urine sample, indicates that the HAART has not been effective, and an amount of NGAL protein in the second urine sample that is less than the amount of NGAL protein in the first urine sample, indicates that the HAART has been effective.

In some embodiments, the present invention provides a method for determining whether a subject has HIV associated nephropathy (HIVAN), the method comprising measuring the amount of NGAL protein in the urine of the subject, and measuring the amount of a second protein in the urine of the subject, wherein the second protein is selected from the group consisting of, clusterin, KIM-1, interleukin-6, selectin (e.g. P-selectin), endothelin-1, and TIMP-1, and wherein an amount of NGAL protein in the urine of the subject that is more than about 8-fold higher than the amount of the second protein in the urine of the subject, indicates that the subject has HIVAN.

In other embodiments, the present invention provides a method for determining whether a subject has HIV associated nephropathy (HIVAN), the method comprising measuring the amount of NGAL protein in the urine of the subject; and measuring the amount of Socs3 protein in the urine of the subject, wherein an amount of NGAL protein in the urine of the subject that is more than about 4-fold higher than the amount of Socs3 protein in the urine of the subject, indicates that the subject has HIVAN.

In further embodiments, the present invention provides a method for determining whether a subject has HIV associated nephropathy (HIVAN), the method comprising determining the amount of NGAL protein or NGAL mRNA in the kidney of the subject; and determining the amount of a second protein or mRNA in the kidney of the subject, wherein the second protein or mRNA is selected from the group consisting of clusterin, KIM-1, interleukin-6, selectin (e.g. P-selectin), endothelin-1, and TIMP-1, and wherein an amount of NGAL protein or mRNA in the kidney of the subject that is more than about 8-fold higher than the amount of the second protein or mRNA in the kidney of the subject, indicates that the subject has HIVAN.

In another embodiment, the present invention provides a method for determining whether a subject has HIV associated nephropathy (HIVAN), the method comprising determining the amount of NGAL protein or mRNA in the kidney of the subject; and determining the amount of a Socs3 protein or mRNA in the kidney of the subject, wherein an amount of NGAL protein or mRNA in the kidney of the subject that is more than about 4-fold higher than the amount of Socs3 protein or mRNA in the kidney of the subject, indicates that the subject has HIVAN.

In a further embodiment, the present invention provides a method for determining whether a subject has HIV associated nephropathy (HIVAN), the method comprising determining the localization of NGAL protein or NGAL mRNA in the kidney of the subject, wherein localization of NGAL mRNA or NGAL protein in microcysts and/or in microcystic tubules, but not in non-cystic tubules, indicates that the subject has HIVAN.

These and other embodiments are described in the following sections of the applications, including the Detailed Description, Examples, Claims, and Drawings.

### BRIEF DESCRIPTION OF THE FIGURES

FIGS. 1A-C are graphs depicting the gene expression profile of HIV1-tg mice compared to wild-type littermates. FIG. 1A is a graph showing NGAL gene expression (raw data) from microarray studies. FIG. 1B is a graph showing Ngal gene expression in GCRMA normalized chips. FIG. 1C is a graph demonstrating the results from RealTime PCR of NGAL compared to wildtype as control (in log10 scale).

FIGS. 2A-2B are microscope images of the kidney showing HIV1-tg histopathology. FIG. 2A are HIV1-tg bright-field images that reveals necrosis in the cortical region (Top Panel, right image) and the accumulation of free Fe in the proximal tubules shown in blue (Bottom Panel). FIG. 2B are higher magnification microscope images using Prussian blue staining and Ngal staining in situ. (from left to right). Dark/blue staining in the cortex, mainly in the proximal tubules. Center image, Ngal expression in the S3 segment of the proximal tubule and the collecting ducts (dark/purple). Right image, Ngal expression in the collecting ducts (dark/purple).

FIG. 2C. Western blot (left) and protein gel (right) images depicting NGAL detection.

FIG. 3 is a graph showing NGAL levels in the general population, controls with HIV, HIVAN patients not on HAART, and HIVAN patients on HAART. Urinary levels of neutrophil gelatinase-associated lipocalin (NGAL) in the general population (n=411, data from previous study), 4 control patients with HIV but no HIVAN, 2 patients with biopsy-proven HIVAN without HAART therapy, and 2 patients with biopsy-proven HIVAN but not on HAART therapy. Data are mean±standard deviation.

FIG. 4 A-C. FIG. 4A presents a table listing details of the samples used for uNGAL studies. FIG. 4B presents a graph depicting uNGAL levels in HIV+ patients as compared to non-HIV+ patients. FIG. 4C depicts a Western Blot of human uNGAL in HIVAN and control patients.

FIG. 5 provides microscope images of the kidney showing HIV1-tg histopathology. The images are at 10× magnification. Prussian blue stain and Ngal in situ staining was used.

FIG. 6. uNGAL levels displayed as a function of eGFR in patients with CKD due to HIVAN, non-HIVAN FSGS, and diabetic and membranous Nephropathies. There was no correlation between the GFR and uNGAL in the HIVAN population (r=0.082, P=0.8) or the patients with diabetic nephropathy (r=−0.348, P=0.4), but a significant correlation existed between GFR and uNGAL for both non-HIVAN FSGS(r=−0.753, P=0.03) and membranous nephropathy (r=−0.665, P=0.00). All data were log-transformed.

FIG. 7. (A) Acute kidney injury associated genes, hyphoxia-associated genes, and NGAL expression in kidneys of 8-wk-old HIVAN and age- and sex-matched WT littermate mice. (B) Table of AKI and hypoxia genes.

FIG. 8. Induction of uNGAL (A) NGAL real-time PCR in TgFVB kidneys from 3-, 6-, and 8-wk-old mice. Data were normalized for NGAL expression in age- and sex-matched WT FVB/N littermate mice. (B) Coomasie blue stained gels and immunoblots of uNGAL in 3-, 6-, and 8-wk-old TgFVB and WT littermate mice. uNGAL increased between 6 and 8 wk in TgFVB mice, whereas proteinuria remained constant. (C) Sections of 8-wk-old TgFVB mouse kidneys. From left to right: Prussian blue staining demonstrates iron accumulation in cortical proximal tubules. Aquaporin-2 immunocytochemistry marks medullary collecting ducts while in situ hybridization in an adjacent section reveals NGAL expression (dark/blue) in dilated collecting ducts (asterisk represents dilated tubules). Higher power demonstrates cast formation (periodic acid—Schiff stain, dark/purple) and NGAL expression (dark/blue) in two adjacent sections. (D) Increasing number of cysts per unit area in TgFVB kidneys with aging. TgFVB differed significantly from WT kidneys (n=14, P=0.00074 at 4 wk; n=14, P=0.001 at 8 wk).

FIG. 9. Immunoblots showing monomeric uNGAL expression in HIV patients. Three HIVAN patients and three HIV positive patients with other glomerulonephritis' representative of our cohort are shown. Human recombinant NGAL standards (ng) are shown.

FIG. 10. ROC Curves demonstrating the utility of uNGAL to diagnose HIVAN among HIV-positive patients with proteinuria compared to other markers.

### DETAILED DESCRIPTION

### Abbreviations & Definitions

The abbreviation “NGAL” refers to Neutrophil Gelatinase Associated Lipocalin. NGAL is also referred to in the art as human neutrophil lipocalin, siderocalin, a-micropglobulin related protein, Scn-NGAL, lipocalin 2, 24p3, superinducible protein 24 (SIP24), uterocalin, and neu-related lipocalin. These alternative names for NGAL may be used interchangeably herein. Unless stated otherwise, the term “NGAL”, as used herein, includes any NGAL protein, fragment, or mutant that is expressed in the kidney, such as any NGAL protein, fragment, or mutant that is expressed in the kidney in response to HIV infection, and which can be detected in a bodily fluid such as urine, including monomeric NGAL. In some embodiments the NGAL protein is wild-type human NGAL.

The abbreviation “uNGAL” refers to NGAL in the urine.

The abbreviation “HIVAN” refers to HIV-associated nephropathy.

The abbreviation “CKD” refers to chronic kidney disease.

The abbreviation “AKI” refers to acute kidney injury.

The abbreviation “ESRD” refers to end-stage renal disease.

The abbreviation “FSGS” refers to focal segmental glomerulocsclerosis.

The abbreviations “HIV-Tg” and “TgFVB” are used interchangeably herein to refer to mice containing an HIV transgene, as described in Example 1, Example 2, and in Kopp et al. (1992); PNAS; 89: p 1577-1581.

The abbreviations “GCRMA” and “GC-RMA” refer to GC content-robust microarray averaging.

The abbreviation “uL-FABP” refers to urinary liver fatty acid binding protein.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### DESCRIPTION

The present invention is based, in part, on a series of surprising discoveries which are described more fully in the Examples section of the present application. For example, the present invention is based, in part, on the surprising discovery that levels of NGAL protein in the urine of patients with HIV associated nephropathy or “HIVAN” are much higher than the levels of NGAL in the urine of both HIV-positive and HIV-negative patients with other forms of chronic kidney disease, e.g. the levels of NGAL protein in the urine of patients with HIVAN can exceed about 100 μg/g creatinine or more as demonstrated in Examples 2 and 3 and in Tables 1 to 4. The present invention is also based, in part, on the surprising discovery that levels of NGAL protein in the urine of patients with HIVAN are much higher than could have been predicted based on the extent of the kidney disease and the level of impairment of kidney function. For example, as demonstrated in Example 2, the levels of NGAL in the urine of HIVAN patients do not merely mirror other markers of kidney disease or impaired kidney function, such as lower glomerular filtration rates (GFR), higher serum creatinine levels, and proteinuria. As such, the level of NGAL found in the urine of HIVAN patients, e.g. a level of NGAL protein in the urine that exceed about 120 μg/g creatinine, is not a general indicator of kidney disease or kidney failure, but is an indicator of HIVAN in particular.

Accordingly, in one aspect of the invention, the presence of levels of NGAL protein in the urine that exceed about 120 μg/g creatinine can be used to diagnose HIVAN and to distinguish HIVAN from other forms of kidney disease, in both HIV-positive and HIV-negative patients. This is particularly useful because prior to the present invention, a definitive diagnosis of HIVAN required a kidney biopsy. Kidney biopsies are costly, are performed by trained doctors, require sophisticated hospital facilities, require analysis by trained pathologists to look for the presence of microcysts and other histological features of HIVAN, and are associated with a risk of kidney infection and potentially kidney loss. Furthermore, because the microcysts and other features that are indicative of HIVAN can be located deep within the kidney medulla, as opposed to being present in the more superficial cortical layers, a deep kidney biopsy sample is needed. The present invention provides a solution to the problem of needing of kidney biopsy to diagnose HIVAN by providing simple, non-invasive, and relatively inexpensive alternatives to kidney biopsy for the diagnosis of HIVAN. The methods of the invention will be useful throughout the world, but particularly in developing nations, such as African nations, in which the rate of HIV is high and the facilities for biopsy are limited. The methods of the invention can also be used in conjunction with biopsy to aid in the definitive diagnosis of HIVAN. These and other aspects of the invention are described herein.

In one embodiment, the present invention provides methods for determining whether a subject has HIV associated nephropathy (HIVAN), comprising measuring the amount of NGAL protein in a bodily fluid of the subject, wherein an amount of NGAL protein that exceeds a threshold level, such as a threshold level of about 100 μg/g creatinine, or about 105 μg/g creatinine, or about 110 μg/g creatinine, or about 115 μg/g creatinine, or about 120 μg/g creatinine, or about 121 μg/g creatinine, or about 121.5 μg/g creatinine, or about 122 μg/g creatinine, or about 123 μg/g creatinine, or about 125 μg/g creatinine, or about 130 μg/g creatinine, or about 125 μg/g creatinine, or about 130 μg/g creatinine, or about 135 μg/g creatinine, or about 140 μg/g creatinine, or about 145 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, indicates that the subject has HIVAN

In another embodiment, the present invention provides methods for distinguishing between HIV associated nephropathy (HIVAN) and non-HIVAN kidney disease in a subject. Such methods comprise measuring the amount of NGAL protein in a bodily fluid of the subject, wherein an amount of NGAL protein in the urine that exceeds a threshold level, such as a threshold level of about 100 μg/g creatinine, or about 105 μg/g creatinine, or about 110 μg/g creatinine, or about 115 μg/g creatinine, or about 120 μg/g creatinine, or about 121 μg/g creatinine, or about 121.5 μg/g creatinine, or about 122 μg/g creatinine, or about 123 μg/g creatinine, or about 125 μg/g creatinine, or about 130 μg/g creatinine, or about 125 μg/g creatinine, or about 130 μg/g creatinine, or about 135 μg/g creatinine, or about 140 μg/g creatinine, or about 145 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, indicates that the subject has HIVAN and an amount of NGAL protein in the urine that is less than that threshold level, such as a threshold level of about 100 μg/g creatinine, indicates that the subject has HIVAN a non-HIVAN kidney disease.

The above methods can be particularly useful for the early detection of HIVAN, for example, before the onset of symptoms of HIVAN, e.g. in an HIV-positive patient who is at risk for developing HIVAN. Accordingly, in one aspect, the above methods be used to diagnose HIVAN in a subject who is not exhibiting signs of kidney disease, for example in an HIV-positive patient.

In another embodiment, the present invention provides a method for monitoring the progression of HIVAN in a subject, the method comprising measuring the amount of NGAL protein in a first bodily fluid sample taken from the subject and a second bodily fluid sample that is taken from the subject at a later period in time, wherein an amount of NGAL protein in the second sample that exceeds the amount of NGAL protein in the first sample, indicates that the HIVAN is worsening, and an amount of NGAL protein in the second sample that is less than the amount of NGAL protein in the first sample, indicates that the HIVAN is improving. In one embodiment, the first sample can be taken before the initiation of therapy for kidney disease or anti-viral therapy, and the second sample can be taken after the initiation of such therapy. In another embodiment, both samples can be taken after the initiation of therapy for kidney disease or anti-viral therapy. Thus, such methods can be used to monitor the effect of therapy, such as therapy for kidney disease and/or anti-viral therapy, on the progression of HIVAN in a subject. In some embodiments, such methods can be used to monitor the efficacy of highly active antiretroviral therapy (HAART) in the subject.

In yet another embodiment, the present invention provides a solution to the problem of determining whether a subject is a candidate for treatment with highly active antiretroviral therapy (HAART), the method comprising measuring the amount of NGAL protein in a bodily fluid from the subject, wherein an amount of NGAL protein that exceeds a threshold level, such as a threshold level of about 100 μg/g creatine, or about 105 μg/g creatinine, or about 110 μg/g creatinine, or about 115 μg/g creatinine, or about 120 μg/g creatinine, or about 121 μg/g creatinine, or about 121.5 μg/g creatinine, or about 122 μg/g creatinine, or about 123 μg/g creatinine, or about 125 μg/g creatinine, or about 130 μg/g creatinine, or about 125 μg/g creatinine, or about 130 μg/g creatinine, or about 135 μg/g creatinine, or about 140 μg/g creatinine, or about 145 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, indicates that the subject has is a candidate for treatment with HAART. In other embodiments, such methods also comprise subsequently administering HAART to the subject.

In yet another embodiment, the present invention provides a method for determining whether a subject has AIDS, the method comprising measuring the amount of NGAL protein in a bodily fluid from the subject, wherein an amount of NGAL protein that exceeds threshold level, such as a threshold level of about 100 μg/g creatine, or about 105 μg/g creatinine, or about 110 μg/g creatinine, or about 115 μg/g creatinine, or about 120 μg/g creatinine, or about 121 μg/g creatinine, or about 121.5 μg/g creatinine, or about 122 μg/g creatinine, or about 123 μg/g creatinine, or about 125 μg/g creatinine, or about 130 μg/g creatinine, or about 125 μg/g creatinine, or about 130 μg/g creatinine, or about 135 μg/g creatinine, or about 140 μg/g creatinine, or about 145 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, indicates that the subject has AIDS.

In another embodiment, the present invention provides methods of assessing the present and ongoing kidney status in a mammalian subject afflicted with or at a risk of developing HIV associated nephropathy (HIVAN), and with worsening HIVAN, by detecting the quantity (e.g., determining the level) of NGAL in urine. The invention also provides a method of monitoring the effectiveness of a treatment for HIVAN by determining the level of NGAL in urine before and in particular after the treatment. The properties and characteristics of NGAL as a biomarker allow for its use in this manner for the early detection of HIVAN or changes in HIVAN status.

In another embodiment, the present invention provides a method for the early detection of HIV associated nephropathy (HIVAN) in a mammal, comprising the steps of: i) providing a sample of a body fluid obtained from a mammalian subject that is not experiencing HIVAN, the body fluid selected from the group consisting of urine, plasma, and serum; ii) detecting (e.g., determining) the level of NGAL in the sample (e.g., using an antibody against NGAL); and iii) evaluating the HIVAN status of the subject, based on the level of NGAL in the sample. The evaluation of HIVAN status can be used to determine whether the nephropathy is stable, or progressing (progressive renal disease). The method also provides an evaluation of the renal status as a progressive or worsening renal nephropathy with only a single sampling and assay.

Another aspect of the invention provides a method for the detection of any change in HIV associated nephropathy (HIVAN) status of a mammal, comprising the steps of: i) obtaining a first sample of a body fluid from a mammalian subject that is not experiencing HIVAN, the body fluid selected from the group consisting of urine, plasma, and serum; ii) detecting (e.g., determining) the level of NGAL in the first sample (e.g., using an antibody against NGAL); iii) obtaining at least one subsequent sample of the body fluid from the subject a period of time after obtaining the first sample; iv) detecting (e.g., determining) the level of NGAL in the at least one subsequent sample (e.g., using an antibody against NGAL); and v) evaluating the HIVAN status of the subject, based on comparing the level of NGAL in the at least one subsequent sample to the level of NGAL in the first sample, wherein a higher level of NGAL in the subsequent sample is an indication of a worsening nephropathy status in the subject (e.g., and potentially of a worsening HIVAN), and a reduced level of NGAL in the subsequent sample is an indication of an improving nephropathy status in the subject (e.g., and potentially of an improving HIVAN).

Another aspect of the invention provides a method of monitoring the effectiveness of a treatment for HIV associated nephropathy (HIVAN) in a mammal, comprising the steps of: i) obtaining a baseline sample of a body fluid from a mammalian subject experiencing HIVAN, the body fluid selected from the group consisting of urine, plasma, and serum; ii) detecting (e.g., determining) the level of NGAL in the baseline sample (e.g., using an antibody against NGAL); iii) providing at least one treatment for the chronic renal injury to the subject; iv) obtaining at least one post-treatment sample of the body fluid from the subject; v) detecting (e.g., determining) the level of NGAL in the post-treatment sample (e.g., using an antibody against NGAL); and vi) evaluating the effectiveness of the treatment, based on comparing the level of NGAL in the post-treatment sample to the level of NGAL in the baseline sample

A further aspect of the invention provides a method of identifying the extent of HIV associated nephropathy (HIVAN) in a mammal over time, comprising the steps of: i) obtaining at least one first sample of a body fluid at a first time from a mammalian subject that is not experiencing HIVAN, the body fluid selected from the group consisting of urine, plasma, and serum; ii) detecting (e.g., determining) the level of NGAL in the first sample (e.g., using an antibody against NGAL); iii) obtaining at least one subsequent sample of the body fluid at a time which is subsequent to the first time from the subject that is not experiencing HIVAN; iv) detecting (e.g., determining) the level of NGAL in the at least one subsequent sample (e.g., using an antibody against NGAL); and v) determining the extent of the nephropathy in the subject over time, based on comparing the level of NGAL in the at least one subsequent sample to the level of NGAL in the first sample, and the time period between obtaining the first sample and the at least one subsequent sample.

According to the methods of the invention, such as those described above, the bodily fluid can be any sample in which NGAL can be detected, including, but not limited to, blood, serum, or urine. In some embodiments the bodily fluid is urine.

Also according to the methods of the invention, the subject can be any mammalian subject that is susceptible to infection with an immunodeficiency virus and in which that virus causes a nephropathy. In some embodiments the subjects are rodents, such as mice. In some embodiments the subjects are human subjects. The subjects can have already been diagnosed as HIV-positive or may not have been diagnosed as HIV-positive. For example, in some embodiments of the invention, the detection of a level of NGAL in a bodily fluid, such as urine, that exceeds a threshold level such as a threshold level of about 100 μg/g creatine, or about 105 μg/g creatinine, or about 110 μg/g creatinine, or about 115 μg/g creatinine, or about 120 μg/g creatinine, or about 121 μg/g creatinine, or about 121.5 μg/g creatinine, or about 122 μg/g creatinine, or about 123 μg/g creatinine, or about 125 μg/g creatinine, or about 130 μg/g creatinine, or about 125 μg/g creatinine, or about 130 μg/g creatinine, or about 135 μg/g creatinine, or about 140 μg/g creatinine, or about 145 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, can be the first indication that the subject has HIV. That is the diagnosis of HIVAN can be made in a subject who has not yet been confirmed to be HIV positive, for example by standard HIV testing methods.

In certain embodiments, the NGAL protein detected and/or measured in the methods of the present invention has an amino acid sequence as defined by one of the following GenBank accession numbers, NP_{—}005555 (human NGAL), CAA67574 (human NGAL), P80188 (human NGAL), AAB26529 (human NGAL), P11672 (mouse NGAL), P30152 (rat NGAL), AAI132070 (mouse NGAL), AAI132072 (mouse NGAL), AAH33089 (human NGAL), and CAA58127 (human NGAL), or is a homolog, variant, derivative, fragment, or mutant thereof, and/or has at least 80% sequence identity, e.g., 85%, 90%, 95%, 98% or 99% sequence identity, with one of the above sequences.

In certain embodiments of the invention, it can be desirable to use a positive control for the detection of NGAL. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell. 10, 1033-1043; Goetz et al. (2000) Biochemistry 39, 1935-1941; and Mori, et al. (2005) J. Clin Invest. 115, 610-621, the contents of which are hereby incorporated by reference.

As described above, in certain embodiments, the present invention provides methods for determining whether a subject has HIV associated nephropathy (HIVAN), comprising measuring the amount of NGAL protein in a bodily fluid of the subject, wherein an amount of NGAL protein that exceeds a threshold level indicates that the subject has HIVAN. The threshold level can also be selected, for example based on reviewing the data provided in the Examples and Tables 1 and 2, so that the threshold level is sufficiently high that it is more likely than not that a subject having that level of NGAL will have HIVAN as opposed to any other form of kidney disease, such as any other form of chronic kidney disease. Any threshold level that will allow for the selective diagnosis of HIVAN can be chosen. For example, the threshold level can be about 100 μg/g creatinine, or about 105 μg/g creatinine, or about 110 μg/g creatinine, or about 115 μg/g creatinine, or about 120 μg/g creatinine, or about 121 μg/g creatinine, or about 121.5 μg/g creatinine, or about 122 μg/g creatinine, or about 123 μg/g creatinine, or about 125 μg/g creatinine, or about 130 μg/g creatinine, or about 125 μg/g creatinine, or about 130 μg/g creatinine, or about 135 μg/g creatinine, or about 140 μg/g creatinine, or about 145 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, such that a urinary NGAL measurement greater than one of these threshold amounts indicates that the subject has HIVAN. Conversely, a urinary NGAL measurement that is less than a certain threshold amount, for example less than about 100 μg/g creatinine or less than about 150 μg/g creatinine, indicates that the subject does not have HIVAN but may have some other form of kidney disease, such as some other form of kidney disease.

Thus, in one embodiment, a urinary NGAL measurement of greater than about 75 μg/g creatinine indicates that the subject has HIVAN. In another embodiment, a urinary NGAL measurement of greater than about 100 μg/g creatinine, or about 105 μg/g creatinine, or about 110 μg/g creatinine, or about 115 μg/g creatinine, or about 120 μg/g creatinine, or about 121 μg/g creatinine, or about 121.5 μg/g creatinine, or about 122 μg/g creatinine, or about 123 μg/g creatinine, or about 125 μg/g creatinine, or about 130 μg/g creatinine, or about 125 μg/g creatinine, or about 130 μg/g creatinine, or about 135 μg/g creatinine, or about 140 μg/g creatinine, or about 145 μg/g creatinine, indicates that the subject has HIVAN. In another embodiment, a urinary NGAL measurement of greater than about 150 μg/g creatinine indicates that the subject has HIVAN. In another embodiment, a urinary NGAL measurement of greater than about 175 μg/g creatinine indicates that the subject has HIVAN. In another embodiment, a urinary NGAL measurement of greater than about 200 μg/g creatinine indicates that the subject has HIVAN. In another embodiment, a urinary NGAL measurement of greater than about 225 μg/g creatinine indicates that the subject has HIVAN. In another embodiment, a urinary NGAL measurement of greater than about 250 μg/g creatinine indicates that the subject has HIVAN. In another embodiment, a urinary NGAL measurement of greater than 275 μg/g creatinine indicates that the subject has HIVAN. In another embodiment, a urinary NGAL measurement of greater than about 300 μg/g creatinine indicates that the subject has HIVAN. In another embodiment, a urinary NGAL measurement of greater than about 325 μg/g creatinine indicates that the subject has HIVAN. In another embodiment, a urinary NGAL measurement of greater than about 350 μg/g creatinine indicates that the subject has HIVAN. In another embodiment, a urinary NGAL measurement of greater than about 375 μg/g creatinine indicates that the subject has HIVAN. In another embodiment, a urinary NGAL measurement of greater than about 400 μg/g creatinine indicates that the subject has HIVAN. In another embodiment, a urinary NGAL measurement of greater than about 425 μg/g creatinine indicates that the subject has HIVAN. In another embodiment, a urinary NGAL measurement of greater than about 450 μg/g creatinine indicates that the subject has HIVAN. In another embodiment, a urinary NGAL measurement of greater than about 475 μg/g creatinine indicates that the subject has HIVAN. In another embodiment, a urinary NGAL measurement of greater than about 500 μg/g creatinine indicates that the subject has HIVAN. In another embodiment, a urinary NGAL measurement of greater than about 525 μg/g creatinine indicates that the subject has HIVAN. In another embodiment, a urinary NGAL measurement of greater than about 550 μg/g creatinine indicates that the subject has HIVAN. In another embodiment, a urinary NGAL measurement of greater than about 575 μg/g creatinine indicates that the subject has HIVAN. In another embodiment, a urinary NGAL measurement of greater than about 600 μg/g creatinine indicates that the subject has HIVAN. In another embodiment, a urinary NGAL measurement of greater than about 625 μg/g creatinine indicates that the subject has HIVAN. In another embodiment, a urinary NGAL measurement of greater than about 650 μg/g creatinine indicates that the subject has HIVAN. In another embodiment, a urinary NGAL measurement of greater than about 675 μg/g creatinine indicates that the subject has HIVAN. In another embodiment, a urinary NGAL measurement of greater than about 700 μg/g creatinine indicates that the subject has HIVAN. In another embodiment, a urinary NGAL measurement of greater than about 725 μg/g creatinine indicates that the subject has HIVAN. In another embodiment, a urinary NGAL measurement of greater than about 750 μg/g creatinine indicates that the subject has HIVAN. In another embodiment, a urinary NGAL measurement of greater than about 775 μg/g creatinine indicates that the subject has HIVAN. In another embodiment, a urinary NGAL measurement of greater than about 800 μg/g creatinine indicates that the subject has HIVAN. In another embodiment, a urinary NGAL measurement of greater than about 825 μg/g creatinine indicates that the subject has HIVAN. In another embodiment, a urinary NGAL measurement of greater than about 850 μg/g creatinine indicates that the subject has HIVAN. In another embodiment, a urinary NGAL measurement of greater than about 900 μg/g creatinine indicates that the subject has HIVAN. In another embodiment, a urinary NGAL measurement of greater than about 925 μg/g creatinine indicates that the subject has HIVAN. In another embodiment, a urinary NGAL measurement of greater than about 950 μg/g creatinine indicates that the subject has HIVAN. In another embodiment, a urinary NGAL measurement of greater than about 975 μg/g creatinine indicates that the subject has HIVAN. In another embodiment, a urinary NGAL measurement of greater than about 1000 μg/g creatinine indicates that the subject has HIVAN. In another embodiment, a urinary NGAL measurement of greater than about 1500 μg/g creatinine indicates that the subject has HIVAN. In another embodiment, a urinary NGAL measurement of greater than about 2000 μg/g creatinine indicates that the subject has HIVAN. In another embodiment, a urinary NGAL measurement of greater than about 2500 μg/g creatinine indicates that the subject has HIVAN. In another embodiment, a urinary NGAL measurement of greater than about 3000 μg/g creatinine indicates that the subject has HIVAN. In another embodiment, a urinary NGAL measurement of greater than about 3500 μg/g creatinine indicates that the subject has HIVAN. In another embodiment, a urinary NGAL measurement of greater than about 4000 μg/g creatinine indicates that the subject has HIVAN.

In other embodiments, a urinary NGAL measurement that falls within a range can indicate that the subject has HIVAN. In one embodiment, a urinary NGAL measurement in the range of from about 100 μg/g creatinine, or about 105 μg/g creatinine, or about 110 μg/g creatinine, or about 115 μg/g creatinine, or about 120 μg/g creatinine, or about 121 μg/g creatinine, or about 121.5 μg/g creatinine, or about 122 μg/g creatinine, or about 123 μg/g creatinine, or about 125 μg/g creatinine, or about 130 μg/g creatinine, or about 125 μg/g creatinine, or about 130 μg/g creatinine, or about 135 μg/g creatinine, or about 140 μg/g creatinine, or about 145 μg/g creatinine, to about 4000 μg/g creatinine indicates that the subject has HIVAN. In one embodiment, a urinary NGAL measurement in the range of from about 150 μg/g creatinine to about 4000 μg/g creatinine indicates that the subject has HIVAN. In one embodiment, a urinary NGAL measurement in the range of from about 175 μg/g creatinine to about 4000 μg/g creatinine indicates that the subject has HIVAN. In one embodiment, a urinary NGAL measurement in the range of from about 200 μg/g creatinine to about 4000 μg/g creatinine indicates that the subject has HIVAN. In one embodiment, a urinary NGAL measurement in the range of from about 225 μg/g creatinine to about 4000 μg/g creatinine indicates that the subject has HIVAN. In one embodiment, a urinary NGAL measurement in the range of from about 250 μg/g creatinine to about 4000 μg/g creatinine indicates that the subject has HIVAN. In one embodiment, a urinary NGAL measurement in the range of from about 275 μg/g creatinine to about 4000 μg/g creatinine indicates that the subject has HIVAN. In one embodiment, a urinary NGAL measurement in the range of from about 300 μg/g creatinine to about 4000 μg/g creatinine indicates that the subject has HIVAN. In one embodiment, a urinary NGAL measurement in the range of from about 325 μg/g creatinine to about 4000 μg/g creatinine indicates that the subject has HIVAN. In one embodiment, a urinary NGAL measurement in the range of from about 350 μg/g creatinine to about 4000 μg/g creatinine indicates that the subject has HIVAN. In one embodiment, a urinary NGAL measurement in the range of from about 375 μg/g creatinine to about 4000 μg/g creatinine indicates that the subject has HIVAN. In one embodiment, a urinary NGAL measurement in the range of from about 400 μg/g creatinine to about 4000 μg/g creatinine indicates that the subject has HIVAN. In one embodiment, a urinary NGAL measurement in the range of from about 425 μg/g creatinine to about 4000 μg/g creatinine indicates that the subject has HIVAN. In one embodiment, a urinary NGAL measurement in the range of from about 450 μg/g creatinine to about 4000 μg/g creatinine indicates that the subject has HIVAN. In one embodiment, a urinary NGAL measurement in the range of from about 475 μg/g creatinine to about 4000 μg/g creatinine indicates that the subject has HIVAN. In one embodiment, a urinary NGAL measurement in the range of from about 500 μg/g creatinine to about 4000 μg/g creatinine indicates that the subject has HIVAN. In one embodiment, a urinary NGAL measurement in the range of from about 525 μg/g creatinine to about 4000 μg/g creatinine indicates that the subject has HIVAN. In one embodiment, a urinary NGAL measurement in the range of from about 550 μg/g creatinine to about 4000 μg/g creatinine indicates that the subject has HIVAN. In one embodiment, a urinary NGAL measurement in the range of from about 575 μg/g creatinine to about 4000 μg/g creatinine indicates that the subject has HIVAN. In one embodiment, a urinary NGAL measurement in the range of from about 600 μg/g creatinine to about 4000 μg/g creatinine indicates that the subject has HIVAN. In one embodiment, a urinary NGAL measurement in the range of from about 625 μg/g creatinine to about 4000 μg/g creatinine indicates that the subject has HIVAN. In one embodiment, a urinary NGAL measurement in the range of from about 650 μg/g creatinine to about 4000 μg/g creatinine indicates that the subject has HIVAN. In one embodiment, a urinary NGAL measurement in the range of from about 675 μg/g creatinine to about 4000 μg/g creatinine indicates that the subject has HIVAN. In one embodiment, a urinary NGAL measurement in the range of from about 700 μg/g creatinine to about 4000 μg/g creatinine indicates that the subject has HIVAN. In one embodiment, a urinary NGAL measurement in the range of from about 725 μg/g creatinine to about 4000 μg/g creatinine indicates that the subject has HIVAN. In one embodiment, a urinary NGAL measurement in the range of from about 750 μg/g creatinine to about 4000 μg/g creatinine indicates that the subject has HIVAN. In one embodiment, a urinary NGAL measurement in the range of from about 775 μg/g creatinine to about 4000 μg/g creatinine indicates that the subject has HIVAN. In one embodiment, a urinary NGAL measurement in the range of from about 800 μg/g creatinine to about 4000 μg/g creatinine indicates that the subject has HIVAN. In one embodiment, a urinary NGAL measurement in the range of from about 825 μg/g creatinine to about 4000 μg/g creatinine indicates that the subject has HIVAN. In one embodiment, a urinary NGAL measurement in the range of from about 850 μg/g creatinine to about 4000 μg/g creatinine indicates that the subject has HIVAN. In one embodiment, a urinary NGAL measurement in the range of from about 875 μg/g creatinine to about 4000 μg/g creatinine indicates that the subject has HIVAN. In one embodiment, a urinary NGAL measurement in the range of from about 900 μg/g creatinine to about 4000 μg/g creatinine indicates that the subject has HIVAN. In one embodiment, a urinary NGAL measurement in the range of from about 925 μg/g creatinine to about 4000 μg/g creatinine indicates that the subject has HIVAN. In one embodiment, a urinary NGAL measurement in the range of from about 950 μg/g creatinine to about 4000 μg/g creatinine indicates that the subject has HIVAN. In one embodiment, a urinary NGAL measurement in the range of from about 975 μg/g creatinine to about 4000 μg/g creatinine indicates that the subject has HIVAN. In one embodiment, a urinary NGAL measurement in the range of from about 1000 μg/g creatinine to about 4000 μg/g creatinine indicates that the subject has HIVAN.

It should be noted that, in each of the embodiments directed to a diagnosing HIVAN based on a urinary NGAL measurement that falls within a range, the upper limit of the range can be adjusted. For example, instead of an upper limit of about 4000 μg/g creatinine, the upper limit of each of the above ranges can be about 3500 μg/g creatinine, or about 3000 μg/g creatinine, or about 2500 μg/g creatinine, or about 2000 μg/g creatinine, or about 1500 μg/g creatinine, or about 1000 μg/g creatinine.

It should also be noted that, in each of the embodiments directed to diagnosing HIVAN, or directed to determining whether a subject is a candidate for treatment with HAART, or directed to diagnosing HIV, the threshold levels of NGAL and the NGAL ranges that can be used in making such a diagnosis or determination can be any of the threshold levels or ranges specified herein. For example, the same threshold levels and ranges disclosed herein as being levels that indicate that a subject has HIVAN (such as those in paragraphs [0064] and [0065]), may also be used to determine whether a subject is a candidate for treatment with HAART, or to diagnose HIV.

It should also be noted that, although the amounts of NGAL described herein are generally referred to in terms of the amount by mass of NGAL relative to the amount by mass of creatinine, e.g. NGAL μg/g creatinine, NGAL can also be measured and/or represented in other units, including, but not limited to measurements of the amount of NGAL by mass (e.g. in nanograms or micrograms), or measurements of the amount of NGAL by concentration (e.g. in ng/mL), or any other units, and it should be understood that amounts of NGAL measured and/or represented in other units can be equivalent to the amounts and ranges described herein in terms of μg/g creatinine The present invention is not limited to methods that comprise measuring NGAL and also measuring creatinine and/or to methods that comprise calculating the amount of NGAL in a sample of bodily fluid in terms of the number of micrograms per gram of creatinine For example, an amount of NGAL that is represented herein as 100 μg/g creatinine, can also be represented in terms of, and encompasses, alternative measurements/units that correspond to the same amount of NGAL, e.g. the same amount of NGAL expressed in terms of mass (e.g. ng), or in terms of concentration (e.g. ng/mL) or in any other units. One of skill in the art can readily make the necessary conversions between units.

In certain embodiments, other biomarkers can be assessed in order to determine whether a subject has HIVAN. For example, the present invention provides that subjects with HIVAN can also have an elevated level of uL-FABP Thus, in some embodiments the present invention provides methods for determining whether a subject has HIVAN comprising measuring the amount of uL-FABP in the urine of the subject. For example, In one embodiment, the present invention provides methods for determining whether a subject has HIV associated nephropathy (HIVAN), comprising measuring the amount of uL-FABP protein in a bodily fluid of the subject, wherein an amount of uL-FABP protein that exceeds a threshold level, such as a threshold level of about 90 μg/g creatinine, or about 95 μg/g creatinine, or about 100 μg/g creatinine, or about 105 μg/g creatinine, or about 107 μg/g creatinine, or about 109 μg/g creatinine, or about 111 μg/g creatinine, or about 113 μg/g creatinine, or about 115 μg/g creatinine, or about 116 μg/g creatinine, or about 116.2 μg/g creatinine, or about 117 μg/g creatinine, or about 118 μg/g creatinine, or about 120 μg/g creatinine, or about 125 μg/g creatinine, or about 130 μg/g creatinine, or about 135 μg/g creatinine, or about 140 μg/g creatinine, or about 145 μg/g creatinine, or about 150 μg/g creatinine, or about 175 μg/g creatinine, or about 200 μg/g creatinine, or about 225 μg/g creatinine, or about 250 μg/g creatinine, or about 275 μg/g creatinine, or about 300 μg/g creatinine, or about 325 μg/g creatinine, or about 350 μg/g creatinine, or about 375 μg/g creatinine, or about 400 μg/g creatinine, or about 425 μg/g creatinine, or about 450 μg/g creatinine, or about 500 μg/g creatinine, indicates that the subject has HIVAN.

In certain embodiments, other biomarkers can be assessed in addition to NGAL in order to determine whether a subject has HIVAN. For example, the present invention provides that, in addition to having a high level of urinary NGAL, subjects with HIVAN can also have one or more of: (i) proteinuria, (ii) a low CD4 count, (iii) a high viral load (i.e. a high HIV viral load), (iv) a high serum creatinine level, and (v) a high uL-FABP level. Thus, in some embodiments the present invention provides methods for determining whether a subject has HIVAN comprising measuring the amount of NGAL in the urine of the subject and also (i) determining whether the subject has proteinuria, (ii) determining the subject's CD4 count, (iii) determining the subject's viral load, (iv), determining the subjects serum creatinine level, and/or (v) determining the subject's uL-FABP level.

For example, in one embodiment, the combination of a high urinary NGAL measurement, e.g. an amount of NGAL in the urine that exceeds one of the threshold amounts described herein, together with a determination that the subject has a high uL-FABP level, e.g an amount of L-FABP in the urine that exceeds one of the threshold levels described herein, indicates that the subject can have HIVAN.

For example, in one embodiment, the combination of a high urinary NGAL measurement, i.e. a level an amount of NGAL in the urine that exceeds one of the threshold amounts described herein, together with a determination that the subject has proteinuria indicates that the subject can have HIVAN. The presence of any amount of protein (typically albumin) in the urine, in combination with a high urinary NGAL level, can be indicative of HIVAN. For example, the amount of protein (typically albumin) in the urine can be greater than about 0.1 mg/dl, or greater than about 0.2 mg/dl, or greater than about 0.3 mg/dl, or greater than about 0.4 mg/dl, or greater than about 0.5 mg/dl, or greater than about 0.6 mg/dl, or greater than about 0.7 mg/dl, or greater than about 0.8 mg/dl, or greater than about 0.9 mg/dl, or greater than about 1.0 mg/dl, or greater than about 1.1 mg/dl, or greater than about 1.2 mg/dl, or greater than about 1.3 mg/dl. Methods of determining whether a subject has proteinuria are well known in the art and are routinely performed by medical professionals, for example using standard laboratory tests or using urine dipstick methods. Most proteinuria tests are based on the detection of albumin in the urine, and/or by determining the level of albumin in the urea as a function of the urine creatinine level, e.g. determining the urine albumin-to-creatinine ratio (UACR). Any proteinuria test can be used in conjunction with the methods of the present invention.

In another embodiment, the combination of a high urinary NGAL measurement, e.g. a level an amount of NGAL in the urine that exceeds one of the threshold amounts indicated herein, together with a CD4 count that is less than about 300 cells/mm³, or less than about 250 cells/mm³, or less than about 200 cells/mm³, or less than about 175 cells/mm³, indicates that the subject may have HIVAN. Methods of determining the CD4 count of a subject are well known in the art and are routinely performed by medical professionals. Any means for determining a subject's CD4 count can be used in conjunction with the methods of the present invention, including, for example, methods based on using an antibody to CD4 in conjunction with flow cytometry. Some methods for determining CD4 counts are described in “Absolute CD4 T-cell counting in resource-poor settings: direct volumetric measurements versus bead-based clinical flow cytometry instruments”; Dieye et al., Acquir. Immune Defic. Syndr.(2005); May 1; 39: p 32-7; the contents of which are hereby incorporated by reference.

In another embodiment, the combination of a high urinary NGAL measurement, e.g. a level an amount of NGAL in the urine that exceeds one of the threshold amounts indicated above, together with a viral load that is more than about 100,000 IU/ml, or more than about 150,000 IU/ml, or more than about 150,000 IU/ml, or more than about 200,000 IU/ml, or more than about 250,000 IU/ml, or more than about 300,000 IU/ml, indicates that the subject may have HIVAN. Methods of determining the viral load, e.g. HIV viral load, of a subject are well known in the art and are routinely performed by medical professionals. Any method for determining viral load, e.g. HIV viral load, can be used in conjunction with the methods of the present invention. For example, HIV viral load can be determined by reverse transcriptase-polymerase chain reaction (RT-PCR) (for example using the Amplicor™ HIV-1 Monitor Test, Roche Diagnostic Systems, Pleasanton, Calif.), or by nucleic acid sequence-based amplification (NASBA) (for example using the NucliSens™ HIV-1 QT Test, Organon Teknika, Bostel, The Netherlands); or by a signal amplification methodology termed branched chain DNA (bDNA) technique (for example using the Quantiplex™ HIV-1 RNA test, Bayer Diagnostics, Emeryville, Calif.). A review of some HIV viral load tests are described in an article entitled “Molecular-Based Methods for Quantifying HIV Viral Load” by Peter et al. (AIDS Patient Care and STDs (2004), 18(2): p 75-79), the contents of which are hereby incorporated by reference.

In another embodiment, the combination of a high urinary NGAL measurement, i.e. a level an amount of NGAL in the urine that exceeds one of the threshold amounts indicated above, together with a serum creatinine level that is more than about 1 mg/dl, or more than about 1.5 mg/dl, or more than about 2.0 mg/dl or more than about or more than about 2.5 mg/dl, or more than about 3 mg/dl, or more than about 4 mg/dl indicates that the subject may have HIVAN. Methods of determining the serum creatinine level of a subject are well known in the art and are routinely performed by medical professionals. Any method for determining a subject's serum creatinine level can be used in conjunction with the methods of the present invention. A review of some methods for determining serum creatinine levels is provided in: Clin. Biochem. Rev. (2006); 27(4): p 173-184; “Measurement of Serum Creatinine—Current Status and Future Goals” by Peake et al., the contents of which are hereby incorporated by reference.

According to the methods of the invention, samples of a bodily fluid can be obtained and/or tested using any means. For example, methods for collecting, handling and processing urine, blood, serum and plasma, and other body fluids, are well known in the art and can be used in the practice of the present invention. In some embodiments, two or more consecutive or subsequent samples of a body fluid can be taken. Depending upon the circumstances, including the level of NGAL in a sample and the clinical condition of the patient, the subject's body fluid can be sampled daily, or weekly, or within a few weeks, or monthly or within a few months, semi-annually, annually, or within several years, and at any interval in between. Repeat sampling can be done at a period of time after treatment to detect any change in HIVAN status and to examine changes in the extent of chronic renal injury due to HIVAN over time. Sampling need not be continuous, but can be intermittent (e.g., sporadic). In some embodiments, it is not be necessary to obtain and keep a sample of the bodily fluid from the subject. For example, in some embodiments, the subject can urinate onto a test strip, for example a test strip of the type used in pregnancy testing kits. In other embodiments, a sample of bodily fluid, such as blood from a pin prick, can be applied onto a test strip—for example a test strip similar to those used for blood typing. Although generally the sample of a bodily fluid, such as blood or urine, is obtained from a subject and tested by a laboratory or by a medical professional (for example using an NGAL testing kit, e.g. a urine dipstick based kit, or an ELISA based kit), home-testing kits are also within the scope of the present invention. In one aspect, the present invention comprises a kit for performing the methods of the invention, containing, for example, a device for detecting NGAL protein in the urine, and optionally including positive control containing NGAL protein, and optionally including instructions, for example regarding the threshold levels of NGAL above which a diagnosis of HIVAN can be made. The device in such kits can comprise, for example, an ELISA plate, a dipstick to be dipped in a urine sample, or a stick on which the subject should urinate. In some embodiments, such devices are configured such that they give a positive result only if the level of NGAL exceeds a threshold level, such as one of the threshold levels described herein. Methods for making and using such devices are well known in the art. Kits (ELISA kits), antibodies, and other reagents for detection of NGAL are commercially available, e.g. from Bioporto Diagnostics A/S and from R & D Systems.

According to the methods of the invention, the presence and/or amount of NGAL or uL-FABP protein in a bodily fluid, such as urine, can be detected and/or measured using any means known in the art. For example, in one embodiment, NGAL or uL-FABP protein can be detected using antibodies that are specific to NGAL or uL-FABP. Any antibody, such as a monoclonal or polyclonal antibody, that binds to NGAL or uL-FABP can be used. For example, monoclonal antibodies that bind to NGAL are described in “Characterization of two ELISAs for NGAL, a newly described lipocalin in human neutrophils”, Lars Kjeldsen et al., (1996) Journal of Immunological Methods, Vol. 198, 155-16, the contents of which are herein incorporated by reference. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety. To prepare this polyclonal antibody, rabbits were immunized with recombinant gel-filtered NGAL protein. Sera were incubated with GST-Sepharose 4B beads to remove contaminants, yielding the polyclonal antibodies in serum, as described by the applicants in Jun Yang et al., Molecular Cell (2002). Further non-limiting examples of antibodies that can be used to detect NGAL protein in the methods of the invention are also provided in the Examples. Antibodies that bind to NGAL are also available commercially, for example from the Antibody Shop, Copenhagen, Denmark, as HYB-211-01, HYB-211-02, and NYB-211-05. In addition, one of skill in the art can readily produce antibodies that bind to NGAL or uL-FABP, or can have them produced by an antibody production company.

Any method can be used to detect and or measure the levels of NGAL or uL-FABP protein, including, but not limited to, immunohistochemistry-based methods, immuno-blotting based methods, immunoprecipitation-based methods, affinity-column based methods (including immunoaffinity column based methods), ELISA-based methods, other methods in which an NGAL antibody is immobilized on a solid substrate (such as beads), and the like. In some embodiments, methods that enable detection of monomeric NGAL may be used, including, but not limited to, immunoblotting methods. In some such methods the antibody to NGAL or uL-FABP, or a secondary or tertiary antibody that binds directly or indirectly to the NGAL or uL-FABP antibody, can be labeled with a detectable moiety, such as a fluorescent moiety, a radioactive moiety, or a moiety that is an enzyme substrate and can be used to generate a detectable moiety, such as horse radish peroxidase. Such methods are well known in the art and can be used to detect the presence and/or measure the amount of NGAL or uL-FABP in a bodily fluid sample, such as urine, without undue experimentation.

In circumstances where the amount of NGAL or uL-FABP is to be measured, positive controls containing known amounts of NGAL or uL-FABP protein can be used, for example for calibration purposes. NGAL or uL-FABP protein for use as a positive control can be obtained from any source or produced by any method known in the art. For example, NGAL or uL-FABP protein can be recombinantly produced. Methods for the recombinant production of proteins are well known in the art. For example, a nucleotide sequence encoding NGAL or uL-FABP can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell. 10, 1033-1043; Goetz et al. (2000) Biochemistry 39, 1935-1941; and Mori, et al. (2005) J. Clin Invest. 115, 610-621, the contents of which are hereby incorporated by reference.

In other aspects of the invention, a diagnosis of HIVAN can be based upon, or can include, detecting the presence of NGAL or uL-FABP protein or mRNA in the kidney itself, as opposed to in a bodily fluid such as urine, for example by detecting a high level of NGAL or uL-FABP protein or mRNA in the kidney itself, or by detecting a specific localization of NGAL or uL-FABP protein or mRNA within the kidney that is indicative of HIVAN. Such methods can be used alone to diagnose HIVAN, or can be used in conjunction with one or more other methods for HIVAN diagnosis such as the methods described herein for detection of NGAL or uL-FABP in urine or other bodily fluids or standard diagnostic methods based on the examination of kidney biopsy samples.

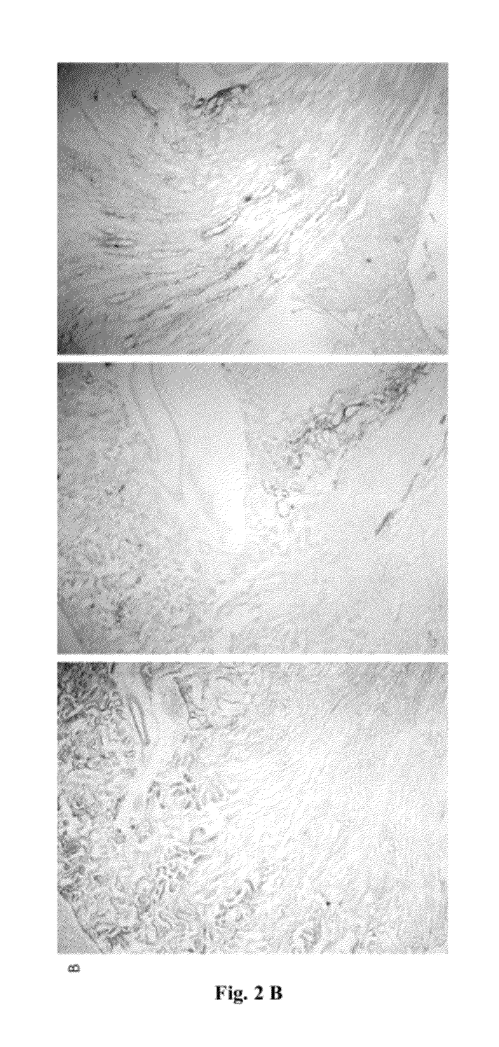

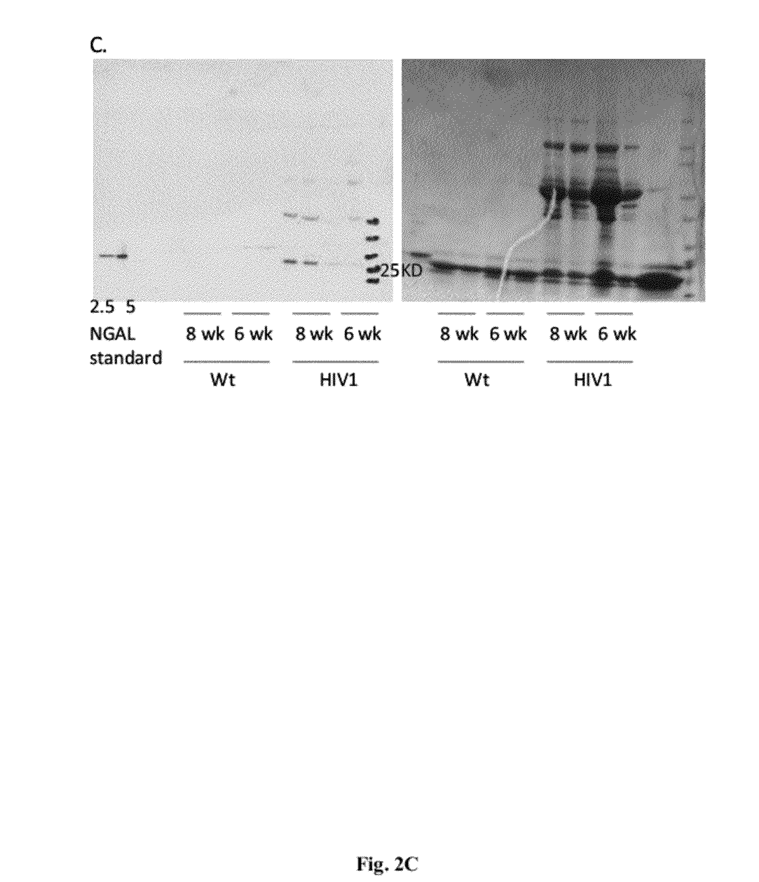

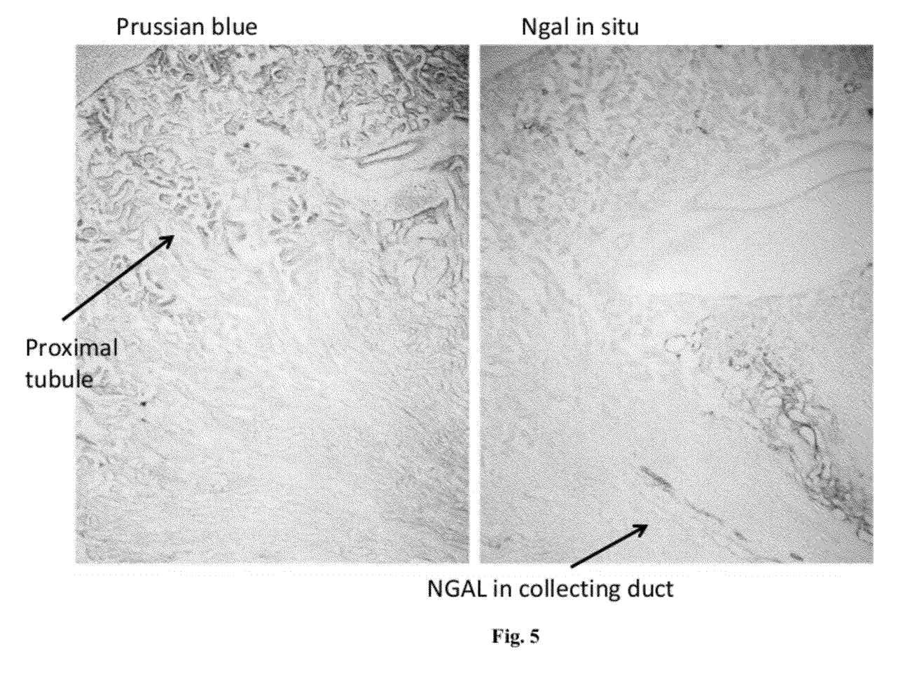

NGAL mRNA and protein is localized in an usual and characteristic pattern in the kidneys of subjects with HIVAN. For example, NGAL protein and mRNA is generally not located in the proximal tubules but is generally located in the collecting ducts, such as aquaporin-2-positive collecting ducts (see Examples and FIGS. 2, 5, and 8). NGAL is also prominently expressed in microcysts in the both the medulla and the cortex, and is prominently expressed in dilated microcystic tubules but not in non-cystic tubules in HIVAN patients (see Examples and FIGS. 2, 5, and 8). This localization of NGAL in the kidneys was unexpected, and indicates that NGAL can be associated with cystogenesis rather than with more global stimuli such as proteinuria, luminal debris, urinary iron, or ischemic injury. Microcyst formation is characteristic of HIVAN. Indeed, currently a diagnosis of HIVAN in an HIV-positive subject is most frequently based on the observation of microcysts and focal sclerosis in a kidney biopsy. Thus, the specific localization of NGAL in the mircrocysts underscores the unique association of NGAL with HIVAN.

Accordingly, in one aspect the present invention provides a method for determining whether a subject has HIVAN, the method comprising determining the localization of NGAL protein or NGAL mRNA in the kidney of the subject, wherein localization of NGAL mRNA or NGAL protein in microcysts, indicates that the subject has HIVAN. In another embodiment, the present invention provides a method for determining whether a subject has HIVAN, the method comprising determining the localization of NGAL protein or NGAL mRNA in the kidney of the subject, wherein localization of NGAL mRNA or NGAL protein in microcystic tubules but not in non-cystic tubules in the kidney, indicates that the subject has HIVAN. Such methods can be performed using a kidney biopsy sample. However, methods for assessing the expression and/or localization of NGAL protein or mRNA in the kidney in situ are also provided by the invention, for example methods wherein, for example, labeled agents that bind to NGAL protein or mRNA are delivered to a subject and can be visualized in vivo, for example using imaging techniques such as CAT scan—based techniques and MRI-based techniques.

Detection of NGAL mRNA or protein in the kidney can be determined using standard techniques and methodologies known to those of skill in the art, for example using samples obtained by biopsy of the kidney. For example, NGAL mRNA can be detected by in situ hybridization using probes specific for NGAL, or by any other method known to be useful for detection of specific mRNAs, including, but not limited to, PCR-based techniques. The sequence of NGAL, including human NGAL, is known in the art. Similarly, sequences of probes and primers that can be used to detect NGAL are known in the art. Furthermore, non-limiting examples of primers and probes that can be used to detect NGAL mRNA are provided in the Examples. In addition, NGAL protein can be detected using antibodies that are specific to NGAL, e.g. monoclonal or polyclonal antibodies can be used. In addition, detection methods that can be used, include, but are not limited to, immunohistochemistry-based methods and the like. Antibodies that are specific to NGAL and that could be used to detect NGAL in the kidneys are known in the art. Monoclonal antibodies for NGAL, are described, for example, in “Characterization of two ELISAs for NGAL, a newly described lipocalin in human neutrophils”, Lars Kjeldsen et al., (1996) Journal of Immunological Methods, Vol. 198, 155-16, herein incorporated by reference in its entirety. Non-limiting examples of antibodies that can be used to detect NGAL protein are provided in the Examples. Antibodies that bind to NGAL are also available commercially, for example from the Antibody Shop, Copenhagen, Denmark, as HYB-211-01, HYB-211-02, and NYB-211-05. Typically, HYB-211-01 and HYB-211-02 can be used with NGAL in both its reduced and unreduced forms. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety. To prepare this polyclonal antibody, rabbits were immunized with recombinant gel-filtered NGAL protein. Sera were incubated with GST-Sepharose 4B beads to remove contaminants, yielding the polyclonal antibodies in serum, as described by the applicants in Jun Yang et al., Molecular Cell (2002).

In other embodiments of the invention, a diagnosis of HIVAN can be based upon an assessment of the amount of NGAL protein or mRNA in a bodily fluid, such as urine, or in the kidney, relative to the amount of one or more other markers. As is illustrated in the Examples, NGAL is the most highly up-regulated gene in the kidney in a mouse model of human HIV/HIVAN. Surprisingly, the level of NGAL expression was around five-fold or more higher than that of the next best gene (suppressor of cytokine signaling 3 (Socs3)), around 10-fold or more higher than a handful of other genes (including clusterin, KIM-1, interleukin-6, selectin (e.g. P-selectin), endothelin-1, and TIMP-1), and around 100-fold or more higher than most other genes tested that have been proven to be involved in acute kidney injury and/or proven to be hypoxia-specific genes (including, but not limited to, the following genes: adrenoedullin, bone morphogenetic protein 7, cAMP responsive element BP1, cysteine rich protein 61, fibroblast growth factor 2, glutathione S-transferase alpha 1, hypoxia inducible factor 1a, heme oxygenase (decycling) 1, insulin-like growth factor 1, insulin-like growth factor 1 receptor, IL18, IL6, N-acetyl galactosaminidase alpha, Na+/H+ exchanger domain 1, phosphodiesterase 4B cAMP, secreted phosphoprotein 1, vimentin, wingless-related integr. site 4, aldolase 1 A isoform, BCL2/1B interacting protein 1 NIP3, carbonic anhydrase 10, carbonic anhydrase 11, carbonic anhydrase 12, carbonic anhydrase 13, carbonic anhydrase 14, carbonic anhydrase 15, carbonic anhydrase 2, carbonic anhydrase 3, carbonic anhydrase 4, carbonic anhydrase 5a, carbonic anhydrase 5b, carbonic anhydrase 7, carbonic anhydrase 8, carbonic anhydrase 9, connective tissue growth factor, chemokine receptor 4, enolase 1 alpha non-neuron, EPO receptor, FMS-like tyrosine kinase 1, heme oxygenase (decycling) 1, lactate dehydrogenase A, nitric oxide synthase 1 neuronal, nitric oxide synthase 1 adaptor, nitric oxide synthase 3 endo, nitric oxide synthase interprot, solute carrier family 2 member 1, transferrin receptor, transferrin, transferrin receptor 2, VEGFa, and xanthine dehydrogenase). This data underscores the dramatic nature of the upregulation of NGAL in HIVAN and its unique ability to be used in methods of diagnosis of HIVAN.

Thus, in one embodiment, the present invention provides a method for determining whether a subject has HIV associated nephropathy (HIVAN), and/or for distinguishing between HIVAN and other forms of kidney disease (both chronic and acute), the method comprising determining the amount of NGAL protein or NGAL mRNA in the kidney of the subject; and determining the amount of a second protein or mRNA in the kidney of the subject, wherein the second protein or mRNA is selected from the group consisting of Socs-3, clusterin, KIM-1, interleukin-6, selectin (e.g. P-selectin), endothelin-1, and TIMP-1, and wherein an amount of NGAL protein or NGAL mRNA in the kidney of the subject that is more than about or more than about 4-fold, or more than about 5-fold, or more than about 6-fold or more than about 7-fold, or more than about 8-fold, or more than about 9-fold or more than about 10-fold higher than the amount of the second protein or mRNA in the kidney of the subject, indicates that the subject has HIVAN.

In one embodiment, the present invention provides a method for determining whether a subject has HIV associated nephropathy (HIVAN), the method comprising determining the amount of NGAL protein or mRNA in the kidney of the subject; and determining the amount of a second protein or mRNA in the kidney of the subject, wherein the second mRNA is selected from the group consisting of clusterin, KIM-1, interleukin-6, selectin (e.g. P-selectin), endothelin-1, and TIMP-1, and wherein an amount of NGAL mRNA in the kidney of the subject that is more than about 7-fold, or more than about 8-fold, or more than about 9-fold, or more than about 10-fold higher than the amount of the second protein or mRNA in the kidney of the subject, indicates that the subject has HIVAN.

In another embodiment, the present invention provides a method for determining whether a subject has HIV associated nephropathy (HIVAN), the method comprising determining the amount of NGAL protein or mRNA in the kidney of the subject; and determining the amount of a KIM-1 protein or mRNA in the kidney of the subject, wherein an amount of NGAL protein or mRNA in the kidney of the subject that is more than about 3-fold, or more than about 4-fold, or more than about 5-fold, or more than about 6-fold, or more than about 7-fold, or more than about 8-fold, or more than about 9-fold, or more than about 10-fold, or more than about 11-fold, or more than about 12-fold higher than the amount of KIM-1 protein or mRNA in the kidney of the subject, indicates that the subject has HIVAN.

In another embodiment, the present invention provides a method for determining whether a subject has HIV associated nephropathy (HIVAN), the method comprising determining the amount of NGAL protein or mRNA in the kidney of the subject; and determining the amount of a Socs3 protein or mRNA in the kidney of the subject, wherein an amount of NGAL protein or mRNA in the kidney of the subject that is more than about 3-fold, or more than about 4-fold, or more than about 5-fold, or more than about 6-fold, or more than about 7-fold, or more than about 8-fold, or more than about 9-fold, or more than about 10-fold, or more than about 11-fold, or more than about 12-fold higher than the amount of Socs3 protein or mRNA in the kidney of the subject, indicates that the subject has HIVAN.

In another embodiment, the present invention provides a method for determining whether a subject has HIV associated nephropathy (HIVAN), the method comprising determining the amount of NGAL protein in a bodily fluid, such as urine, of the subject; and determining the amount of a second protein in a bodily fluid, such as urine, of the subject, wherein the second protein is selected from the group consisting of clusterin, KIM-1, interleukin-6, selectin (e.g. P-selectin), endothelin-1, and TIMP-1, and wherein an amount of NGAL protein in the bodily fluid, such as urine, of the subject that is more than about 7-fold, or more than about 8-fold, or more than about 9-fold, or more than about 10-fold higher than the amount of the second protein, indicates that the subject has HIVAN.

In another embodiment, the present invention provides a method for determining whether a subject has HIV associated nephropathy (HIVAN), the method comprising determining the amount of NGAL protein in a bodily fluid, such as urine, of the subject; and determining the amount of a KIM-1 protein in a bodily fluid, such as urine, of the subject, wherein an amount of NGAL protein in a bodily fluid, such as urine, of the subject that is more than about 7-fold, or more than about 8-fold, or more than about 9-fold, or more than about 10-fold higher than the amount of KIM-1 protein, indicates that the subject has HIVAN.

In another embodiment, the present invention provides a method for determining whether a subject has HIV associated nephropathy (HIVAN), the method comprising determining the amount of NGAL protein in a bodily fluid, such as urine, of the subject; and determining the amount of a Socs3 protein in a bodily fluid, such as urine, of the subject, wherein an amount of NGAL protein in a bodily fluid, such as urine, of the subject that is more than about 3-fold, or more than about 4-fold, or more than about 5-fold higher than the amount of Socs3 protein, indicates that the subject has HIVAN.

In the above embodiments, clusterin, KIM-1, interleukin-6, P-selectin, endothelin-1, TIMP-1, and/or Socs3 mRNA or protein can me measured using the same types of methodologies as described above for measurement of NGAL protein or mRNA, including but not limited to antibody-based detection methods for protein and nucleic acid-based detection methods for mRNA.

Antibodies that bind to clusterin, KIM-1, interleukin-6, P-selectin, endothelin-1, TIMP-1, and Socs3 are known in the art, widely commercially available, and can be used to detect and/or measure each of these proteins. For example, antibodies that bind to clusterin are available from Santa Cruz Biotechnology, Inc. (Santa Cruz, Calif.), ProSci, Inc. (Poway, Calif.), and GenWay Biotech, Inc. (San Diego, Calif.). Antibodies against KIM-1 can be obtained from Novus Biologicals LLC (Littleton, Colo.) and R&D Systems (Minneapolis, Minn.). Antibodies that bind to interleukin-6 are available from Abcam (Cambridge, UK), eBioscience, Inc. (San Diego, Calif.), and Santa Cruz Biotechnology, Inc. (Santa Cruz, Calif.). Antibodies against P-selectin are available from eBioscience, Inc. (San Diego, Calif.), and Santa Cruz Biotechnology, Inc. (Santa Cruz, Calif.), and eBioscience, Inc. (San Diego, Calif.), and Santa Cruz Biotechnology, Inc. (Santa Cruz, Calif.). Abcam (Cambridge, UK), eBioscience, Inc. (San Diego, Calif.), and Santa Cruz Biotechnology, Inc. (Santa Cruz, Calif.), and Novus Biologicals LLC (Littleton, Colo.) supply anti-endothelin-1 antibodies and anti-Socs3 antibodies. Antibodies that bind to TIMP-1 can be obtained from Abcam (Cambridge, UK), GenWay Biotech, Inc. (San Diego, Calif.), and Lab Vision Corp. (Fremont, Calif.).

Similarly, the sequences of clusterin, KIM-1, interleukin-6, P-selectin, endothelin-1, TIMP-1, and Socs3 genes and/or mRNAs are known in the art and can be used to generate primers and/or probes to detect and/or measure each of these mRNAs. For example, the sequence of KIM-1 is provided by Ichimura et al. (J. Biol. Chem., 273:4135-42, 1998), which is hereby incorporated by reference. The sequences of the other molecules are provided by the following GenBank accession numbers: clusterin-AY341244; interleukin-6-NG011640; P-selectin-NG012125; endothelin-1-J05008-TIMP-1 Y09720; and Socs3-BC060858.

The invention is further described by the following non-limiting Examples.

### EXAMPLES

### Example 1

### Urinary Ngal to Diagnose HIVAN

The numbers in parentheses below refer to the references in the numbered list that follows this Example.

Neutrophil Gelatinase-Associated Lipocalin (NGAL) protein is massively upregulated in the renal tubule as a response to acute kidney injury (AKI) in adults (13), children (3), and animals (12, 1, 11, 10). We have previously shown that in the setting of AKI, regardless of AKI etiology, urinary NGAL (uNGAL) expression and excretion into the urine peaks within six to twelve hours of renal injury and precedes the rise in serum creatinine by forty-eight hours (12, 21). It has also been shown previously that NGAL is secreted in chronic kidney injury.

NGAL is a small polypeptide that is protease resistant and consequently readily detected in the urine and serum. In chronic renal tubule cell injury, NGAL protein can typically be detected in direct proportion to the degree and severity of the injury. Incremental increases in kidney or circulating NGAL levels in chronic renal failure patients over a prolonged period of time are diagnostic of worsening kidney disease. This increase in NGAL precedes and correlates with other urinary and circulating indicators of worsening chronic renal failure, such as increased serum creatinine, increased urine protein secretion, and lower glomerular filtration rate (GFR). Concurrently, circulating NGAL is expressed extra-renally into the bloodstream. Typically, NGAL is excreted at a higher concentration in urine than in blood for a particular event.

NGAL has been extensively studied in humans, mice, and rats with normal renal function and in acute renal disease. NGAL was found to be normally secreted into the circulation by the liver and spleen, and it is filtered by the glomerulus and then recovered by the proximal tubule. NGAL is degraded in the lysosomes (from 23 KDa to 14 KDa), and ligands located in the NGAL calyx are released. The capture of circulating, non-kidney NGAL by the proximal tubule is very effective, as little, if any NGAL is found in the urine of normal humans and mice (in humans: filtered load=(21 ng/mL circulating NGAL) X (GFR), whereas urinary NGAL=22 ng/mL. In mice: filtered load=(100 ng/ml circulating NGAL) X (GFR), whereas urinary NGAL=40 ng/ml). Even after massive overload of the NGAL protein by systemic injections of NGAL (1 mg), there is little protein recovered in the urine. The uptake into the proximal tubule likely reflects the action of megalin. This was ascertained in a megalin knockout mouse that contains a marked increase in the injected NGAL in the urine. Only a small amount of degraded NGAL (14 kDa) is found in the urine, reflecting processing within the kidney. The plasma t_{1/2} was ˜10 min, and was likely the result of renal clearance. These data stress the specificity of urinary NGAL (NGAL recovered from urine) as a biomarker of kidney-expressed NGAL.

In the early years of the HIV epidemic, Rao et at reported on a series of HIV patients in New York City who showed rapid progression to ESRD, and a pattern of cFSGS on biopsy (16). HIVAN is a condition classically characterized by proteinuria, azotemia, large kidneys on ultrasound, and cFSGS on biopsy. The incidence of HIV is increasing, with 2.7 million new infections worldwide and 2 million HIV-related deaths in 2007(20). In 2007, there were 4,232 biopsy-proven cases of HIVAN in the US, with a 31.6% mortality rate (14). However, the actual prevalence of HIVAN is estimated to be higher, as only 50% of suspected cases of HIVAN are actually biopsied, and it is estimated that up to 10% of patients with HIV will developed cFSGS, the lesion classically associated with HIVAN (11, 12). The vast majority of the patients with HIVAN are African American, suggesting the disease may have a strong genetic component as well (7) The incidence of HIVAN has reached a relatively steady state since the advent of HAART in the late 1990s (19). Although it remains unclear if initiating HAART therapy and suppressing viral replication after the diagnosis of HIVAN can lead to improvement or stabilization of renal function, data does suggest that early initiation of HAART can prevent the development of HIVAN (15).

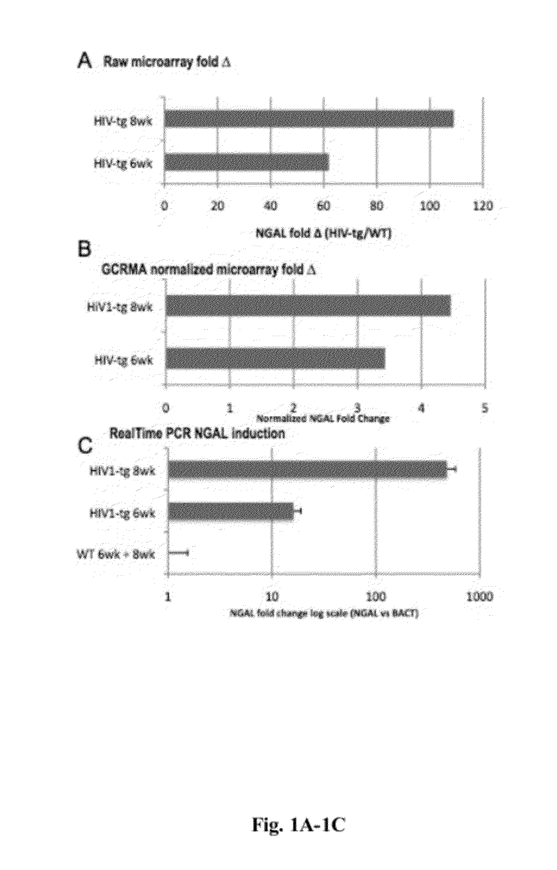

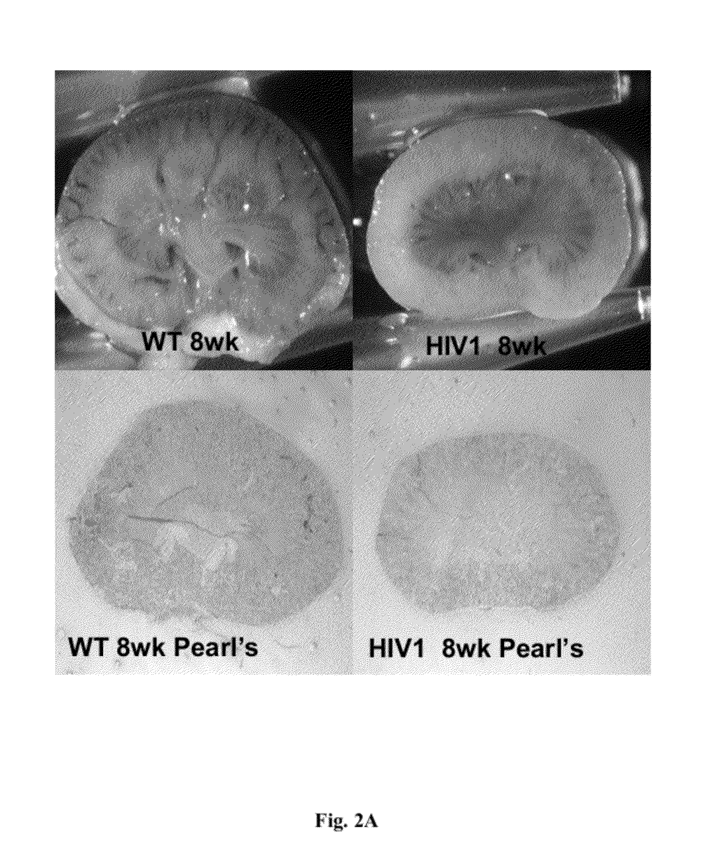

It is well documented that HIV transgenic (HIV-tg) animals have progressive proteinuria due to renal failure, pathologically characterized by a collapsing glomeruli, similar to human cFSGS. To identify genes activated in these events, Affymetrix arrays (Mouse Genome 430 2.0) were used to compare the gene expression profile of HIV1-tg mouse kidneys to its wild-type littermate at both 6 and 8 weeks of age. NGAL was the one of the most up-regulated genes in both the 6 and 8 week-old HIV-tg kidneys out of 39,000 transcripts with a raw fold change of 62 fold and 109 fold (FIG. 1A). After GCRMA normalization of all chips, NGAL was still the most up-regulated with 3.43 fold and 4.46 fold change, respectively (FIG. 1B), and these array data taken together, NGAL upregulation was highly statistically significant (p<1.38E-70). Furthermore, the production of NGAL was confirmed using Realtime PCR (FIG. 1C). Of particular interest was the expression of genes associated with acute kidney injury (AKI) and hypoxia, as HIVAN is a chronic form of renal disease associated with pathological characteristics of AKI. Out of 24 AKI and hypoxia specific genes, the most highly expressed was the gene corresponding to NGAL protein.

To investigate the spatial distribution of NGAL in the kidney, performed in situ hybridization was performed using paraformaldehyde fixed kidneys (FIG. 2B). The data demonstrated that NGAL expression is restricted to the collecting ducts of HIVAN displaying HIV-tg mice, and interestingly, when the adjacent serial section of these kidneys was stained for Prussian Blue—a marker of ferric iron—the NGAL expressing collecting ducts appear to be immediately downstream of the heavily stained Prussian Blue positive tubules (FIGS. 2 A & B). This indicates that NGAL is being activated downstream of the site of injury, possibly due the presence of cellular debris and reactive oxygen species due to iron overload. Renal function of the HIV-tg animals was further evaluated by measuring urinary protein and uNGAL in HIV-tg heterozygotes and their Wt littermates. HIV-tg animals had marked proteinuria at 6 and 8 wks, as previously reported by Kopp et al. (1992); PNAS; 89: p 1577-1581 and they also had increased uNGAL compared to controls (FIG. 2C).

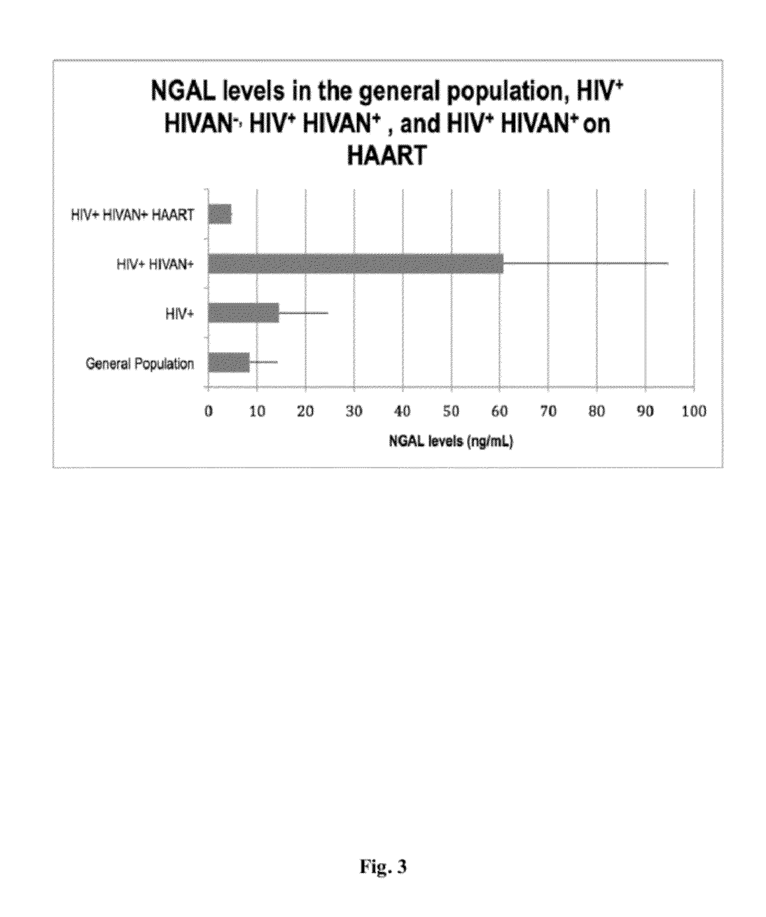

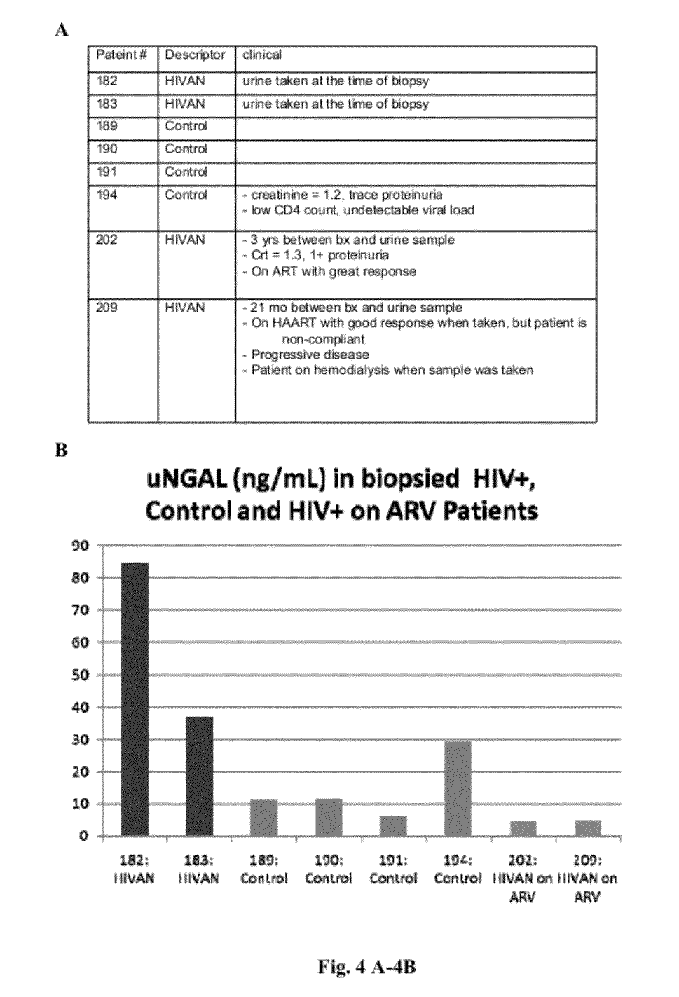

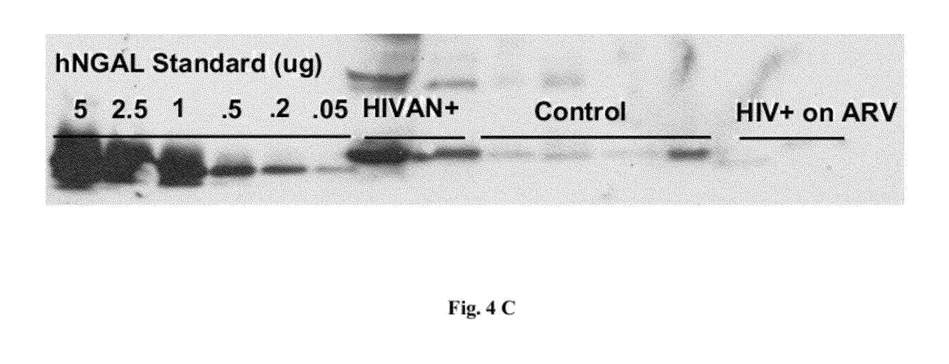

The characteristics of uNGAL expression was investigated in patients infected with HIV in a cross-sectional cohort of patients (some with HIVAN; some with biopsy proven HIVAN and some with HIVAN and treated with HAART). Patients with biopsy proven HIVAN and not on HAART had increased uNGAL excretion (60.7±33.7 ng/mL) compared both to HIVAN patients on HAART (4.7±0.2 ng/mL) and patients with HIV without HIVAN (14.5±10.1 ng/mL) (FIG. 3). All patients on HAART with biopsy proven HIVAN had urine collected after the onset of therapy. These data suggest that elevated uNGAL levels are associated with HIVAN and can be used to indicate the renal response to HAART treatment.

uNGAL may be highly expressed in the setting of HIVAN because this chronic kidney disease is characterized by acute pathological features. The data provided in this application indicates that uNGAL, which is secreted into the urine by the collecting ducts of HIVAN patients, can be used to identify HIVAN. NGAL has been shown to be both highly specific and sensitive for the detection of AKI (13, 18). Pathologically, HIVAN demonstrates prominent tubulointerstitial disease, with marked tubular dilation and focal tubular degenerative changes (4). Additionally, observational data from a series of 635 patients presenting to an inner-city emergency department demonstrated one of the highest NGAL levels in the entire cohort in a patient with fulminant cFSGS.

The development of HAART has reduced the incidence of HIVAN. Unfortunately, HIVAN incidence remains elevated in areas of the world, such as Africa, where HAART is not widely available.(22) Although HIVAN incidence remains elevated in Africa, detection of HIVAN is suboptimal secondary to technological limitations, inability to perform renal biopsy and poor healthcare structures. The present invention provides a solution to such problems because measurement of uNGAL can be simply performed by Western Blot, ELISA or by other urine tests. Therefore, the uNGAL assays of the invention can provide resource poor countries with an improved ability to detect HIVAN and prioritize HAART administration to those patients who may derive a renal benefit.

In conclusion, this Example shows that uNGAL is expressed in the setting of HIVAN and can be used as a biomarker of HIVAN and avert the necessity of renal biopsy. Furthermore, this diagnostic information can permit refined management decisions regarding HAART administration. The presence of uNGAL in HIV+ patients can also permit the ability of early HAART administration to prevent further deterioration in renal function and the developed of ESRD. It is therefore possible that HIVAN is not a late complication of HIV-1 infection, but a late detected condition due to the insensitivity of serum creatinine as an indicator of kidney function. This Example shows that uNGAL is a superior marker of HIVAN and provides prognostic information regarding future kidney function in comparison to the serum creatinine

### Animals

Heterozygous mice transgenic for human immunodeficiency virus (HIV-tg) (6) were bred with FVB/N mice to produce heterozygotes. Hetereozygosity was determined by the presence of cataracts.

### Western Blot

Urine was centrifuged (12,000 rpm, 10 minutes) and supernatants stored at −20 C. Urine hNGAL (10 μg) and mNGAL (1 μl) was quantified by western blots, using non-reducing 4-15% tris-HCL gels (Bio-Rad, Laboratories, Inc. Hercules, Calif.) and monoclonal (1:1000; Antibody Shop, BioPorto Diagnostics, Gentofte, Denmark) or rabbit polyclonal antibodies together with standards (0.2-10 ng) of human recombinant NGAL protein and mouse monoclonal antibodies (1:1000) reproducible to 0.4 ng/lane. Briefly, 10 ml of sample and 3 ml of non-reducing sample buffer were loaded into each lane, and run until the loading buffer reached the bottom of the gel. Gels were then transferred to nitrocellulose transfer membranes (Whatman, Dassel, Germany), blocked with 1% skim milk, and washed overnight in a 1:1000 of primary antibody. The membranes were subsequently washed in TBST, and places in secondary antibody (ECL Anti-mouse IgGHorseradish Peroxidaselinked whole antibody, GE healthcare) for 1 hour. Membranes were then washed with TBST, rinsed with ECL (Amersham ECL Western Blotting Detection Reagent, GE healthcare), and exposed. The extent of expression of NGAL protein was quantified using ImageJ software (National Institute of Mental Health, Bethesda, Md.).

### In Situ Hybridization

Digoxigenin-labeled antisense riboprobes were generated from cDNAs encoding Ngal (exon 1-7, 566 bp) by linearization with XhoI followed by T7 RNA polymerase. Kidneys were collected in ice-cold PBS and fixed overnight at 4° C. in 4% paraformaldehyde (PFA) in 0.1 M phosphate buffer saline (PBS), briefly quenched in 50 mM NH₄Cl, cryoprotected overnight in 30% sucrose PBS and embedded and sectioned (16 μM) in O.C.T. compound. The sections were post-fixed in 4% PFA for 10 min, treated with proteinase K (1 mg/ml) for 3 min, acetylated and prehybridized for 2 hrs, and then hybridized at 68-72° C. overnight. The prehybridization and hybridization solution was 50% formamide, 5′ SSC, 5′ Denhardts, 250 mg/ml baker's yeast RNA (Sigma), and 500 mg/ml herring sperm DNA (Sigma). Sections were washed at 72° C. in 5′ SSC for 5-10 minutes, then at 72° C. in 0.2° SSC for 1 hour and then stained overnight (4° C.) with an anti-digoxigenin antibody coupled with alkaline phosphatase (Boehringer-Mannheim), at a 1:5000 dilution in 0.1 M Tris-HCl, pH 7.5, 0.15 M NaCl, 1% heat inactivated goat serum. Alkaline phosphatase activity was detected using BCIP, NBT (Boehringer-Mannheim) with 0.25 mg/ml levamisole in a humidified chamber for 1-3 days in the dark. Sections were dehydrated and mounted in Permount (Fisher Scientific).

### Prussian Blue Reaction

Frozen sections as described above were rinsed with water for 5 min. Slides were incubated in freshly prepared 2.5% potassium ferrocyanide and 2.5% hydrochloric acid for 20 min at room temperature (RT). Slides were then rinsed in distilled water for 5 min, dehydrated in alcohol, cleared in xylene and coverslipped.

### Total RNA

RNA was isolated with the mirVANA RNA extraction kit (Ambion). Total RNA quantified by a Nanoprop machine (Thermo Scientific, Delaware) and qualified by gel electrophoresis.

### Real-Time PCR

HIV-tg and wild type (WT) controls were prepared according to Bio-Rad SYBR GREEN, iCyclerMyiQ protocols. Target genes, including Ngal, Kim1, and β-actin, utilized respectively: Ngal 116 forward primer 5′-ctcagaacttgatccctgcc-3′ (SEQ ID NO. 1) and NGALa593 reverse 5′-tccttgaggcccagacactt-3′ (SEQ ID NO. 2); Kim1 s46 5′-ccaggcactgtggattcttatgtgg-3′ (SEQ ID NO. 3) and Kim1a630 5′-tgtgacatcctcaggagatacctgg-3′(SEQ ID NO. 4); β-actin415 forward primer 5′-ctaaggccaaccgtgaaaag-3′(SEQ ID NO. 5) and β-actin 696 reverse primer 5′-tctcagctgtggtggtgaag-3′ (SEQ ID NO. 6). The ΔΔCT method was used to calculated fold amplification of transcripts.

### Microarray Analysis

Double-stranded cDNA was synthesized from 7 ug of total RNA extracted from whole kidneys, purified, and in vitro transcription biotin labeled cRNA generated for GeneChip hybridization (GeneChip One-Cycle Target Labeling Kit, Affymetrix). Fragmented biotin labeled cRNA was hybridized to Affymetrix Mouse Genome 430 2.0 GeneChips.

### Statistical Analysis

All data sets GCRMA normalized and absolute mean of 6 and 8 week old data pairs, HIV-tg and WT, normalized difference in signal expression values were generated and Bonferroni corrected where any value p-value greater than 3.17 E-6 is meaningless.

### Patients

Anonymized urine samples from patients with biopsy-proven HIVAN were obtained from Mt Sinai School of Medicine, and the Manhattan HIV Brain Bank (MHBB). Samples from the Mt Sinai School of Medicine were collected at the time of kidney biopsy, and then frozen at −80° C. Samples of patients with HIV but without HIVAN were used as controls. Laboratory personnel were blinded to the status of the patients.

### References for Example 1

1. Barasch, J & Mori, K: Cell biology: iron thievery. Nature, 432: 811-3, 2004.
2. Behar, DM, Shlush, L I, Maor, C, Lorber, M & Skorecki, K: Absence of HIV-associated nephropathy in Ethiopians. Am J Kidney Dis, 47: 88-94, 2006.
3. Bennett, M, Dent, CL, Ma, Q, Dastrala, S, Grenier, F, Workman, R, Syed, H, Ali, S, Barasch, J & Devarajan, P: Urine NGAL predicts severity of acute kidney injury after cardiac surgery: a prospective study. Clin J Am Soc Nephrol, 3: 665-73, 2008.
4. D'Agati, V & Appel, GB: HIV infection and the kidney. J Am Soc Nephrol, 8: 138-52, 1997.
5. He, J C, Husain, M, Sunamoto, M, D'Agati, VD, Klotman, M E, Iyengar, R & Klotman, PE: Nef stimulates proliferation of glomerular podocytes through activation of Src-dependent Stat3 and MAPK1,2 pathways. J Clin Invest, 114: 643-51, 2004.
6. Kaufmann, R, Laroche, D, Buchner, K, Hucho, F, Rudd, C, Lindschau, C, Ludwig, P, Hoer, A, Oberdisse, E, Kopp, J & et al.: The HIV-1 surface protein gp120 has no effect on transmembrane signal transduction in T cells. J Acquir Immune Defic Syndr, 5: 760-70, 1992.
7. Klotman, PE: HIV-associated nephropathy. Kidney Int, 56: 1161-76, 1999.
8. Korgaonkar, S N, Feng, X, Ross, M D, Lu, T C, D'Agati, V, Iyengar, R, Klotman, PE & He, J C: HIV-1 upregulates VEGF in podocytes. J Am Soc Nephrol, 19: 877-83, 2008.
9. Lu, T C, He, J C & Klotman, P E: Podocytes in HIV-associated nephropathy. Nephron Clin Pract, 106: c67-71, 2007.
10. Mishra, J, Ma, Q, Prada, A, Mitsnefes, M, Zahedi, K, Yang, J, Barasch, J & Devarajan, P: Identification of neutrophil gelatinase-associated lipocalin as a novel early urinary biomarker for ischemic renal injury. J Am Soc Nephrol, 14: 2534-43, 2003.
11. Mishra, J, Mori, K, Ma, Q, Kelly, C, Yang, J, Mitsnefes, M, Barasch, J & Devarajan, P: Amelioration of ischemic acute renal injury by neutrophil gelatinase-associated lipocalin. J Am Soc Nephrol, 15: 3073-82, 2004.
12. Mori, K, Lee, HT, Rapoport, D, Drexler, I R, Foster, K, Yang, J, Schmidt-Ott, K M, Chen, X, Li, J Y, Weiss, S, Mishra, J, Cheema, F H, Markowitz, G, Suganami, T, Sawai, K, Mukoyama, M, Kunis, C, D'Agati, V, Devarajan, P & Barasch, J: Endocytic delivery of lipocalin-siderophore-iron complex rescues the kidney from ischemia-reperfusion injury. J Clin Invest, 115: 610-21, 2005.
13. Nickolas, T L, O'Rourke, M J, Yang, J, Sise, M E, Canetta, P A, Barasch, N, Buchen, C, Khan, F, Mori, K, Giglio, J, Devarajan, P & Barasch, J: Sensitivity and specificity of a single emergency department measurement of urinary neutrophil gelatinase-associated lipocalin for diagnosing acute kidney injury. Ann Intern Med, 148: 810-9, 2008.
14. NIHDK: USRDS 2008 Annual Data Report: Atlas of End-Stage Renal Disease in the United States. National Institute of Health, 2008.
15. Post, F A, Campbell, L J, Hamzah, L, Collins, L, Jones, R, Siwani, R, Johnson, L, Fisher, M, Holt, S G, Bhagani, S, Frankel, A H, Wilkins, E, Ainsworth, J G, Larbalestier, N, Macallan, D C, Banerjee, D, Baily, G, Thuraisingham, R C, Donohoe, P, Hendry, B M, Hilton, R M, Edwards, S G, Hangartner, R, Howie, A J, Connolly, J O & Easterbrook, P J: Predictors of renal outcome in HIV-associated nephropathy. Clin Infect Dis, 46: 1282-9, 2008.
16. Rao, T K, Filippone, E J, Nicastri, A D, Landesman, S H, Frank, E, Chen, C K & Friedman, E A: Associated focal and segmental glomerulosclerosis in the acquired immunodeficiency syndrome. N Engl J Med, 310: 669-73, 1984.
17. Rosenstiel, P E, Gruosso, T, Letourneau, A M, Chan, J J, Leblanc, A, Husain, M, Najfeld, V, Planelles, V, D'Agati, V D, Klotman, M E & Klotman, P E: HIV-1 Vpr inhibits cytokinesis in human proximal tubule cells. Kidney Int, 2008.
18. Schmidt-Ott, K M, Mori, K, Li, J Y, Kalandadze, A, Cohen, D J, Devarajan, P & Barasch, J: Dual action of neutrophil gelatinase-associated lipocalin. J Am Soc Nephrol, 18: 407-13, 2007.
19. Selik, R M, Byers, R H, Jr. & Dworkin, M S: Trends in diseases reported on U.S. death certificates that mentioned HIV infection, 1987-1999. J Acquir Immune Defic Syndr, 29: 378-87, 2002.
20. UNAIDS: Report on the Global AIDS Epidemic. UN, 2008.
21. Wagener, G, Jan, M, Kim, M, Mori, K, Barasch, J M, Sladen, R N & Lee, H T: Association between increases in urinary neutrophil gelatinase-associated lipocalin and acute renal dysfunction after adult cardiac surgery. Anesthesiology, 105: 485-91, 2006.
22. Wyatt, C M & Klotman, P E: HIV-1 and HIV-Associated Nephropathy 25 Years Later. Clin J Am Soc Nephrol, 2 Suppl 1: S20-4, 2007.
23. Zhong, J, Zuo, Y, Ma, J, Fogo, A B, Jolicoeur, P, Ichikawa, I & Matsusaka, T: Expression of HIV-1 genes in podocytes alone can lead to the full spectrum of HIV-1-associated nephropathy. Kidney Int, 68: 1048-60, 2005.

### Example 2

### Urinary Ngal Marks Cystic Disease in HIVAN

The numbers in parentheses below refer to the references in the numbered list that follows this Example.

In 2007 alone, there were 2.7 million new infections with HIV and 2 million HIV-related deaths worldwide. (1). An important complication of HIV is a form of kidney disease called HIV-associated nephropathy (HIVAN), which occurs predominantly in patients of African descent. (2-4). The prevalence of HIVAN may be as high as 15% of HIV patients (based upon autopsy data), and 4232 new cases of HIVAN reached ESRD between 2002 and 2006 in the United States. (5).

HIVAN is a rapidly progressive form of chronic kidney disease (CKD) characterized by nephrotic range proteinuria. Kidney biopsies demonstrate histologic abnormalities in both glomeruli and tubules, including collapsing FSGS, podocyte proliferation and dedifferentiation, tubular dilation, microcyst formation, and tubulointerstitial inflammation. (6,7). The pathogenesis is believed to be due to dysregulation of podocytes and tubular epithelia by HIV-1 itself (8,9). Early identification of HIVAN is important because highly active antiretroviral therapy (HAART), corticosteroids, and inhibition of renin-angiotensin may delay disease progression (6,10,11). Nonetheless, because HIV infection may be associated with other glomerular disease, definitive diagnosis of HIVAN requires a kidney biopsy. In fact, half of all patients with presumed HIVAN demonstrated different types of lesions once biopsied (11,12).

Neutrophil gelatinase-associated lipocalin (NGAL) is a 22-kD protein that is markedly upregulated in renal tubules and urine (uNGAL) in response to epithelial damage. (13-15). Expression of NGAL peaks 12 h after acute injury (14,15) but remains elevated if injury is severe (16). In a study of 650 patients presenting to an inner-city emergency department, it was found that a single spot uNGAL test could distinguish ongoing injury from physiologic changes in renal function found in prerenal azotemia and from periods of slow or limited progression found during the course of many types of CKD (“stable CKD”). (13).

Nephrosis and a rapid decline in kidney function characterize HIV-associated nephropathy (HIVAN). Histologically, HIVAN is a collapsing focal segmental glo-merulosclerosis (FSGS) with prominent tubular damage. The expression of neutrophil gelatinase-associated lipocalin (NGAL), a marker of tubular injury, was explored to determine whether this protein can be used for the noninvasive diagnosis of HIVAN. Expression of urinary NGAL was found to be much higher in patients with biopsy-proven HIVAN than in HIV-positive and HIV-negative patients with other forms of chronic kidney disease. In the HIV-transgenic mouse model of HIVAN, NGAL mRNA was abundant in dilated, microcystic segments of the nephron. In contrast, urinary NGAL did not correlate with proteinuria in human or in mouse models. These data show that marked upregulation of NGAL accompanies HIVAN.

To test the association of uNGAL and HIV, a cohort of 13 patients with biopsy-proven HIVAN was examined. Biopsied or autopsied kidneys showed global (41% of nephrons per section), collapsing (19%), and segmental(12%) glomerulosclerosis and tubular atrophy (48%) and microcysts (18%), consistent with HIVAN. This group was compared with 24 race-matched HIV-positive controls with normal kidney function, defined as an estimated GFR (eGFR)>60 ml/min and no evidence of proteinuria. Comparisons were also made to HIV-positive and HIV-negative cohorts with other forms of CKD.

Patients with HIVAN had significantly lower CD4 counts and higher viral loads, serum creatinine levels, and proteinuria compared with HIV-positive, race-matched controls (Table 1). In HIVAN, uNGAL was up-regulated 11-fold in comparison with that of HIV-positive race-matched controls without kidney disease (mean values, 748±1160 μg/g creatinine in HIVAN versus 68±98 μg/g creatinine without HIVAN; P value 0.006). Furthermore, in comparison with HIV-positive patients with CKD of non-HIVAN etiology (CKD, HIV-positive), uNGAL was upregulated approximately fivefold regardless of race-matching (Table 2). Moreover, uNGAL was 34-fold higher in patients with HIVAN compared with HIV-negative patients with CKD secondary to membranous nephropathy (n=16), non-HIVAN FSGS (n=7), or diabetic and hypertensive kidney diseases (n=12) (13). In fact, uNGAL levels in patients with HIVAN were more typical of patients presenting to an inner-city emergency department with acute kidney injury (AKI) (n=30, Table 2) than those in the same emergency department with stable CKD (CKD, HIV-negative, n=106).

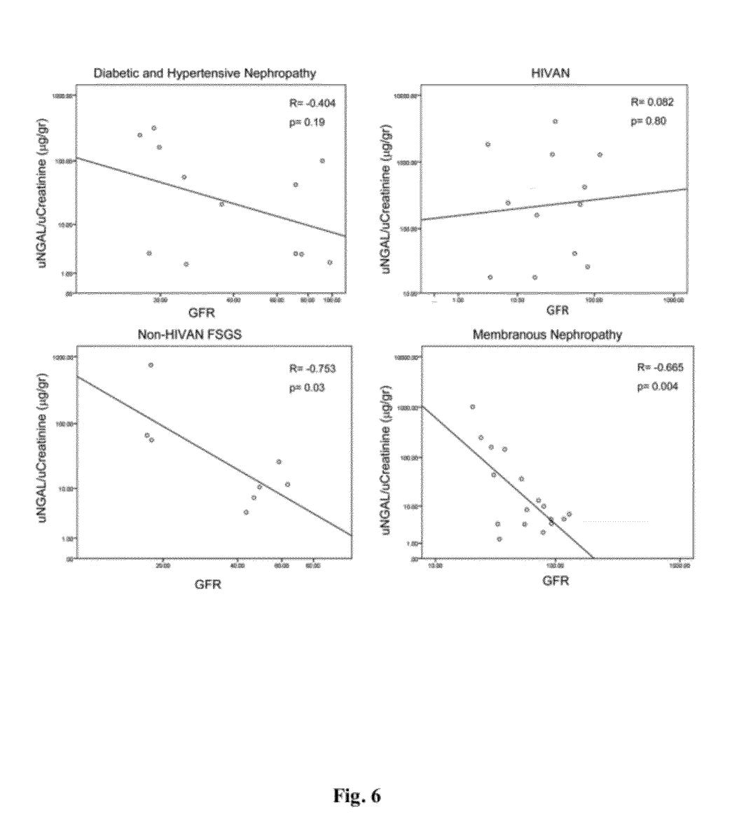

The enhanced expression of uNGAL in HIVAN was not due to higher levels of serum creatinine, lower eGFR, or proteinuria. uNGAL showed no correlation with eGFR in HIVAN (r=0.082, P=0.8), whereas it was inversely related to eGFR in two other forms of CKD (FIG. 6), membranous nephropathy (r=0.665, P=0.004), and FSGS (r=−0.753, P=0.03). In fact, five HIVAN patients with relatively preserved kidney function (serum creatinine <2, mean eGFR 79.4, range 117.35 to 56.22) and limited proteinuria (0.68 g/L, range 0.0 to 3.0 mg/ml) demonstrated markedly elevated levels of uNGAL (mean 401 ng/ml, range 42 to 1285 ng/ml), showing that uNGAL can be expressed early in the course of progressive renal failure due to HIVAN. Consistently, there was no correlation between uNGAL and proteinuria (r=−0.28, P=0.4). In contrast, uNGAL levels were significantly correlated with viral load (R=0.469, P=0.005), and uNGAL was suppressed by HAART (three of the patients with low levels of uNGAL were receiving HAART). In summary, rather than renal failure itself, characteristics of HIVAN appeared to accelerate uNGAL expression. The lack of association between uNGAL and glomerular functional markers (eGFR and proteinuria) showed that HIVAN stimulated uNGAL at a tubular site.

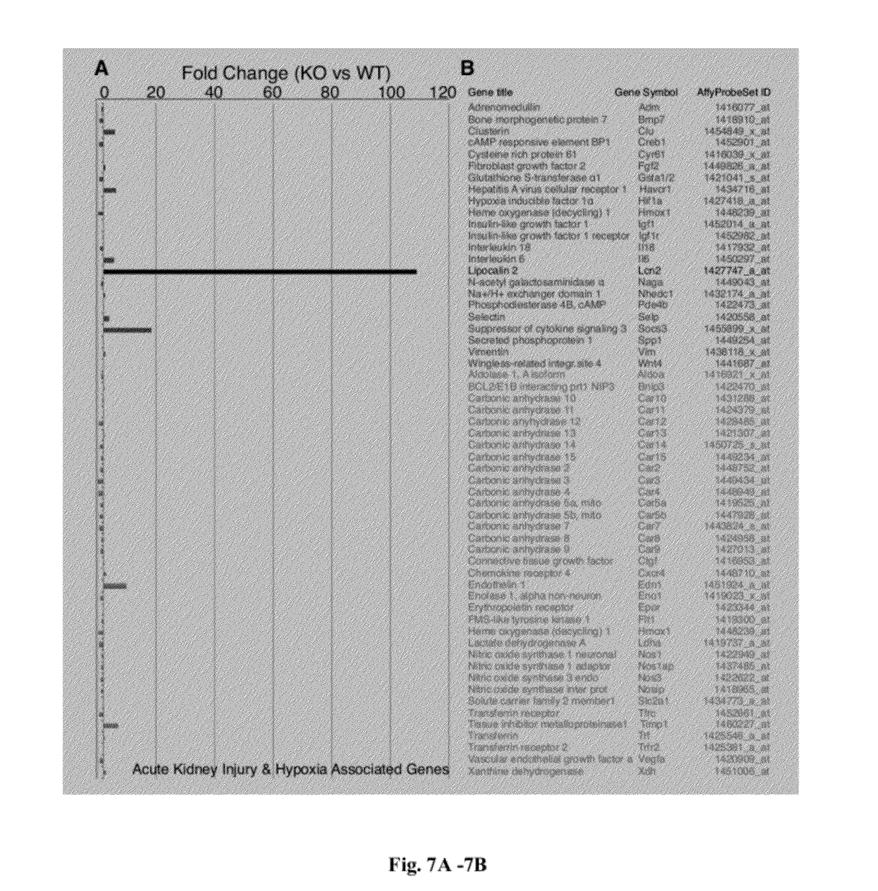

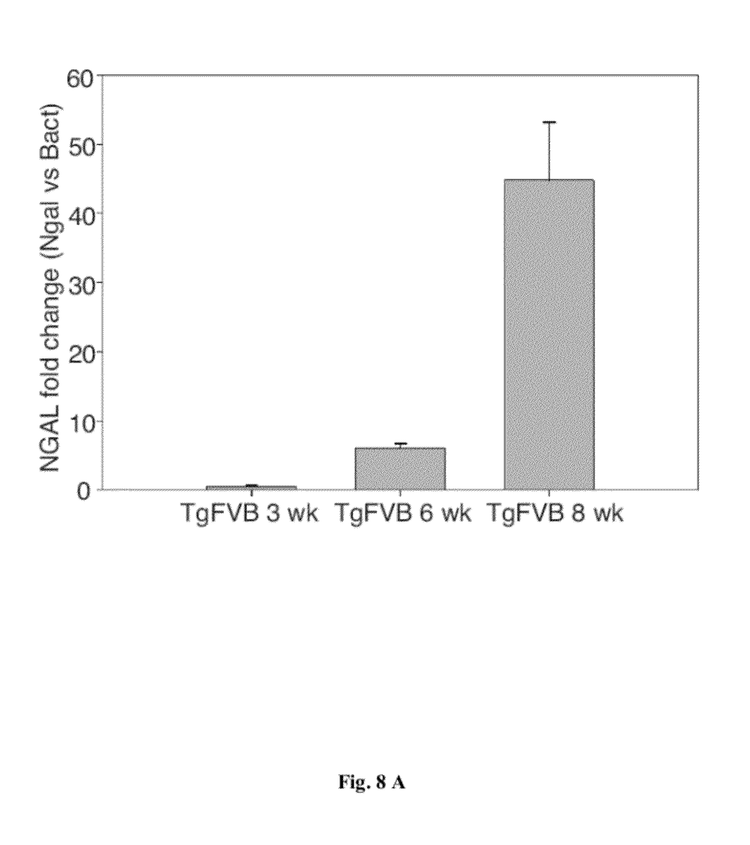

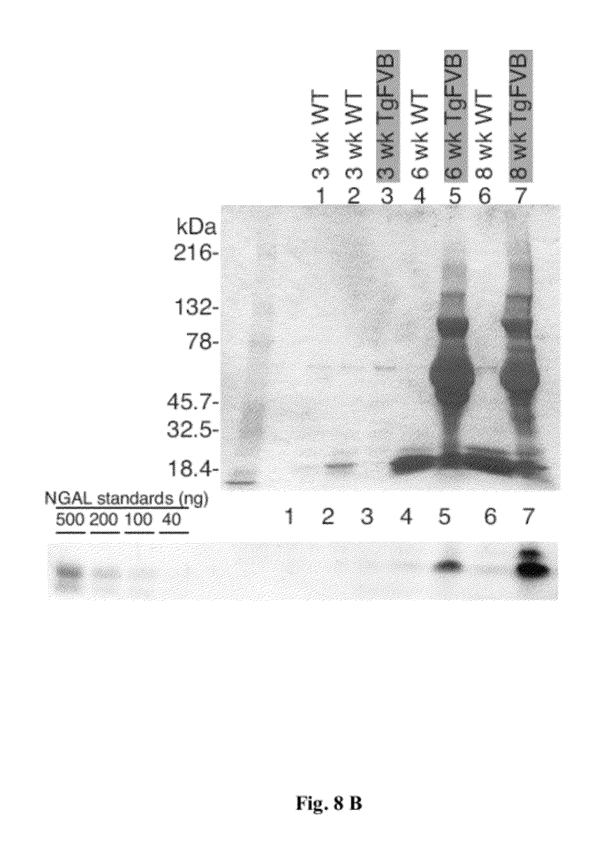

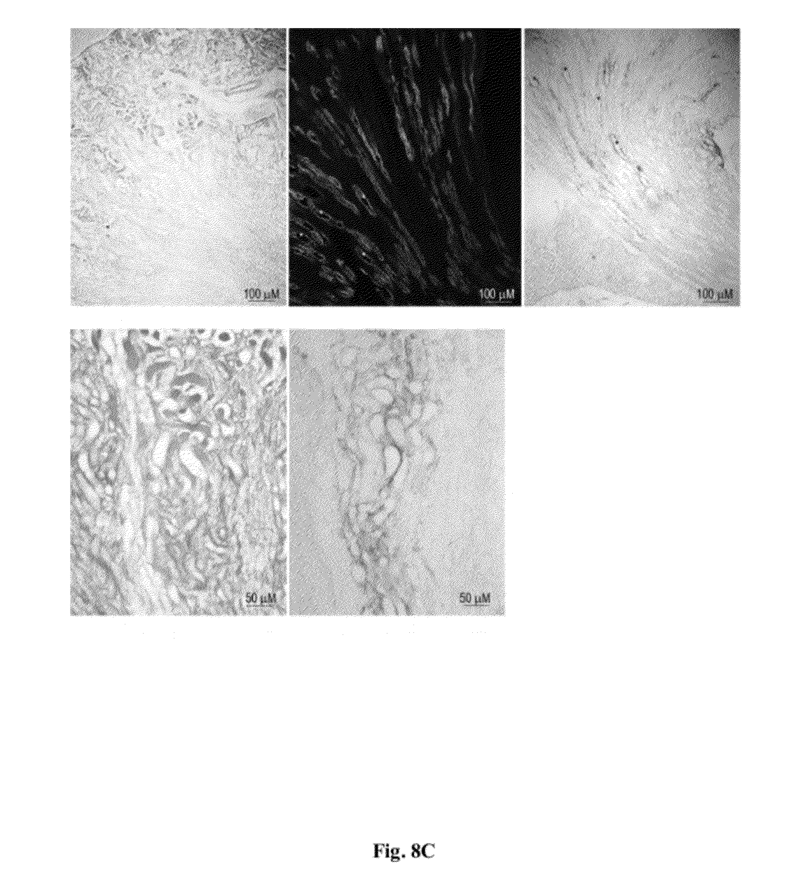

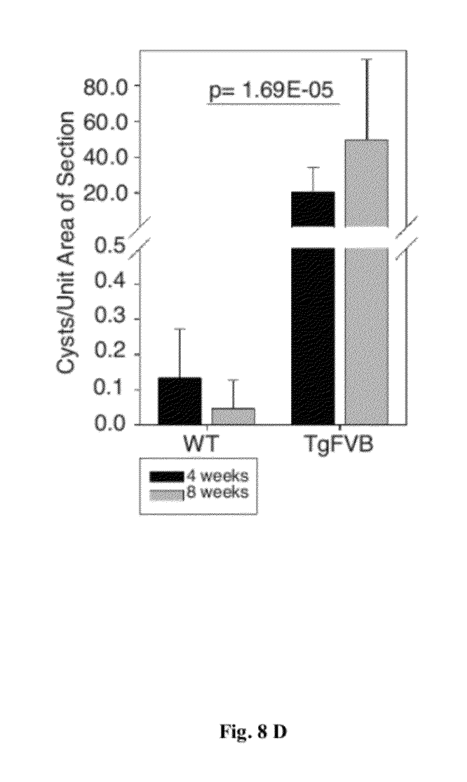

Because HIV-transgenic mice (Tg-FVB) (17) display a syndrome identical to HIVAN (18), NGAL expression was measured in kidneys of Tg-FVB and wild-type (WT) littermates (Affymetrix, Mouse Genome 430 2.0 ml-croarrays; Geo Accession Number Series GSE14221). NGAL was one of the most highly upregulated genes among 39,000 transcripts, demonstrating 62- and 109-fold increases at 6 and 8 wk, respectively (FIG. 7A). After GC content-robust microarray averaging (GC-RMA) normalization of TgFVB and WT samples, NGAL was the most upregulated gene (TgFVB versus WT,P=1.4×1O˜70). In fact, out of 23 proven AKI and 35 proven hypoxia-specific genes, 19 (FIGS. 7, A and B) NGAL was the most differentially expressed gene, fivefold higher than the next best gene and 100-fold higher than most others. The expression of NGAL in HIVAN kidneys was confirmed using real-time-PCR (FIG. 8A) and the presence of NGAL in the urine by immuno-blot (FIG. 8B). NGAL mRNA and uNGAL protein increased over time with the progression of kidney disease (FIGS. 8, A and B).

TgFVB mice excrete as much as 10 g/L of high-molecular-mass proteins commonly seen in other glomerular diseases (FIG. 8B). (17). In addition, as with other types of nephrosis, the proximal tubule demonstrated Prussian-blue-positive iron accumulation, presumably the result of the capture of iron-bearing proteins from the filtrate (FIG. 8C). Given that both proteinuria and iron filtration have been implicated as agents that damage the proximal tubule, (20) NGAL expression at this site was examined. Surprisingly, in situ hybridization showed that NGAL mRNA was not expressed in the proximal tubule, where stainable iron was found, but rather in aquaporin-2-positive collecting ducts of TgFVB mice (FIG. 8C). NGAL was prominently expressed in dilated microcystic tubules rather than homogenously throughout a specific nephron segment, which would be typical of, for example, ischemia-reperfusion injury (16). NGAL mRNA was detected in 39% of microcysts (n=2698 microcysts), in both medulla and cortex. In contrast, NGAL mRNA was not detected in non-cystic tubules. This unexpected finding suggested that NGAL was likely induced by a local stimulus associated with cystogenesis rather than a global stimulus such as proteinuria, luminal debris, urinary iron, or ischemic injury. In this regard, it was notable that, although proteinuria was constant between 6 and 8 wk of life, NGAL expression increased over time, consistent with an increase in cyst number per area of section as these mice age (FIG. 8D).

The invention provides the finding that that uNGAL is highly activated in HIVAN. uNGAL achieved levels more typical of AKI than CKD. The elevated levels of uNGAL may originate from the filtrate, but in our study, there was no correlation between uNGAL and proteinuria in either human or mouse HIVAN. Similarly, in a larger cohort of patients with biopsy-proven CKD of various etiologies, there was no correlation between proteinuria and uNGAL (Pearson correlation, r=0.085, P=0.5, n=88 patients), suggesting that sources of NGAL other than the glomerular filtrate contribute to uNGAL. In HIVAN, the source was identified as cystic tubular epithelia. This finding is consistent with the observation that uNGAL is expressed in patients with poly-cystic kidney disease, (21) especially in those with rapid cyst enlargement.

NGAL was previously identified as a tubulogenic factor, (22) a property supported by studies in cell lines. (23). Likewise, in adult kidney, (14) thyroid cells, (24) and gastric epithelia, (25) NGAL acts as a growth factor, whereas in other cells, such as polycystic-kidney-disease-related renal cysts, NGAL may serve as a proapoptotic factor. (26). A common growth mechanism may underlie these phenomena, perhaps with iron loaded NGAL, (22,24) but exact mechanisms are unclear. Nonetheless, it is striking that proliferation and apoptosis have been noted in HIVAN cysts, (27) and the intensive expression of NGAL at these sites shows that one or both of these activities might be modulated by NGAL.

In conclusion, uNGAL was markedly elevated in patients with biopsy-proven HIVAN and in a mouse model of HIVAN. NGAL was produced by tubular cysts and secreted into the urine. These data show that uNGAL can be useful to monitor the formation of renal tubular cysts and consequently diagnose HIVAN and to distinguish HIVAN from other forms of CKD, such as CKD associated with diabetes or hypertension, or other glomerulopathies presenting in the HIV patient. uNGAL testing cam provide a rationale for biopsy and aggressive HAART therapy to prevent the progression of HIVAN to ESRD. (28).

TABLE 1
Mean values and cohort characteristics for HIV-positive
patients with HIVAN or race-matched controls^{a}

| | | | HIV-Positive | | | |
|---|---|---|---|---|---|---|
| | | | Race-Matched | | | |
| | HIVAN | | Controls | | All Patients | |
| | (n = 13) | | (n = 24) | | (n = 37) | |
| Age (yrs) | 42 | (9.6) | 48 | (10) | 48 | (10) |
| Male (%) | 62 | | 71 | | 68 | |
| Hepatitis B (%) | 10 | | 14 | | 13 | |
| Hepatitis C (%) | 10 | | 45 | | 33 | |
| eGFR(ml/min) | 42 | (37) | 128 | (42)^{b} | 99 | (57) |
| Creatinine (mg/dl) | 5.3 | (6.1) | 0.8 | (0.2)^{b} | 2.3 | (4.0) |
| Proteinuria (mg/dl) | 1.3 | (1.9) | 0.0 | (0.0) | 0.5 | (1.3) |
| CD4 (cells/mm³) | 160 | (258) | 340 | (342)^{c} | 277 | (324) |
| Viral load (IU/ml) | 232,535 | (279,496) | 73,591 | (182,928)^{b} | 121,755 | (224,675) |
| uNGAL (μg/g | 748 | (1160) | 68 | (98)^{b} | 307 | (750) |
| creatinine) | | | | | | |

^{a}Continuous data were log-transformed prior to statistical testing.
^{b}P value < 0.05 for comparison between HIVAN and HIV-positive race-matched controls.
^{c}P value < 0.01 for comparison between HIVAN and HIV-positive race-matched controls.
Data is presented as mean (standard deviation).

The left hand column of Table 1 lists parameters assessed in HIVAN and HIV-positive race-matched control cohorts. These parameters include the mean patient age in the cohort, the % of males in the cohort, the percentage of patients in the cohort that were Hepatitis B or Hepatitis C positive, the mean glomerular filtration rate (eGFR) of the cohort, the mean serum creatinine level of the cohort, the mean proteinuria value of the cohort, the mean HIV viral load of the cohort, and the mean urinary NGAL measurement of the cohort.

The second column of Table 1 provides data for the HIVAN cohort, which comprised 13 patients. The third column of Table 1 provides data for the HIV-positive race matched control cohort, which comprised 24 patients. These patients did not have HIVAN. The final column of Table 1 provides data for the all of the subjects, i.e. the mean values across both the HIVAN cohort and the HIV-positive race matched control cohort. The numbers in parentheses are the standard deviation.

TABLE 2
Median values of kidney injury biomarkers: HIV-positive patients with HIVAN and other cohorts^{a}

| | | | | Serum | | | | Protein/ | |
|---|---|---|---|---|---|---|---|---|---|
| Cohort | | NGAL (μg/g | | Creatinine | | GFR | | Creatinine | |
| No. | Cohort Characteristics | creatinine) | | (mg/dl) | | (ml/min) | | Ratio | |
| 1 | Control, HIV-positive, | 28 | (4-481)^{c} | 0.9 | (0.4-1.2)^{c} | 116 | (83-251)^{c} | 0.0 | (0.0-0.0) |
| | race-matched (n = 24) | | | | | | | | |
| 2 | Control, HIV-positive, | 74 | (5-361) | 0.8 | (0.4-1.0)^{c} | 114 | (83-246)^{c} | 0.0 | (0.0-0.3)^{c} |
| | non-race-matched (n = 14) | | | | | | | | |
| 3 | CKD, HIV-positive, | 231 | (18-4050) | 2.6 | (0.9-18.5) | 30 | (4-117) | 0.6 | (0.0-9.6) |
| | HIVAN (n = 13) | | | | | | | | |
| 4 | CKD, HIV-positive, race- | 88 | (5-374) | 2.1 | (1.1-3.4) | 37 | (24-190) | 0.2 | (0.0-8.2) |
| | matched (n = 6) | | | | | | | | |
| 5 | CKD, HIV-positive, non- | 51 | (19-225) | 1.5 | (0.3-2.2) | 57 | (13-344) | 0.5 | (0.0-9.2) |
| | race-matched (n = 10) | | | | | | | | |
| 6 | CKD, HIV-negative | 12 | (1-344)^{c} | 1.5 | (1-12.6) | 43 | (5-90) | N/A | |
| | (n = 106) | | | | | | | | |
| 7 | CKD, HIV-negative, | 8 | (1-248)^{c} | 1.3 | (0.5-3.2)^{b} | 56 | (21-128) | 4.5 | (1.0-15.3)^{b} |
| | membranous (n = 16) | | | | | | | | |
| 8 | CKD, HIV-negative, FSGS | 19 | (4-67)^{b} | 1.2 | (1.1-3.7) | 73 | (17-63) | 1.9 | (1.0-2.4) |
| | (n = 7) | | | | | | | | |
| 9 | CKD, HIV-negative, | 32 | (2-316)^{b} | 2.7 | (0.7-3.6) | 26 | (18-98) | 2.3 | (0.2-27.9) |
| | diabetic and hypertensive | | | | | | | | |
| | kidney diseases (n = 12) | | | | | | | | |

| | | | | | | | | |
|---|---|---|---|---|---|---|---|---|
| 10 | AKI, all causes (n = 30 | 296 | (11-1833) | 3.9 | (0.7-28.6) | 16 | (2-53) | N/A |

^{a}All data presented as median (range).
N/A, not available.
^{b}P value < 0.05 in comparison with the HIVAN cohort.
^{c}P value < 0.01 in comparison with the HIVAN cohort.

The second column of Table 2 describes the 10 patient cohorts for which NGAL, serum creatinine, GFR, and protein creatinine data is provided. The 10 patient cohorts were as follows:
Cohort 1. Race-matched HIV-positive patients without diagnosed kidney disease and without diagnosed HIVAN
Cohort 2. Non-race-matched HIV-positive patients without diagnosed kidney disease and without diagnosed HIVAN.
Cohort 3. HIV-positive patients with chronic kidney disease (CKD) and diagnosed as having HIVAN.
Cohort 4. Race-matched HIV-positive patients with chronic kidney disease (CKD) and not diagnosed as having HIVAN.
Cohort 5. Non-race-matched HIV-positive patients with chronic kidney disease (CKD) and not diagnosed as having HIVAN.
Cohort 6. HIV-negative patients with chronic kidney disease (CKD).
Cohort 7. HIV-negative chronic kidney disease (CKD) patients with membranous nephropathy.
Cohort 8. HIV-negative chronic kidney disease (CKD) patients with focal segmental glomerulosclerosis (FSGS).
Cohort 9. HIV-negative chronic kidney disease (CKD) patients with diabetic and hypertensive kidney diseases.
Cohort 10. Acute Kidney Injury patients.

Columns three to six of Table 2 provide data (median value with range given in parentheses) for each of the parameters measured, as follows: Third column—urinary NGAL expressed as μg/g creatinine); Fourth column—serum creatinine (expressed as mg/dl); Fifth column—glomerular filtration rate (GFR) (expressed in ml/min); Sixth column—protein/creatinine ratio.

### Patients

Anonymized urine samples from patients with biopsy-proven HIVAN were obtained (29). Urine samples were collected at the time of kidney biopsy (n=2), between 20 and 36 mo after kidney biopsy (n=2), or between 0.3 and 37 mo before autopsy (n=9) and then frozen at −80° C. HIV-positive controls and HIV-positive patients with other biopsy-proven CKD were derived from the same cohorts as HIVAN patients. HIV-positive controls divided into race-matched and non-race-matched groups are presented separately in Table 2. HIV-positive controls had normal kidney function (defined by an estimated Modification of Diet in Renal Disease GFR of >60 ml/min), 30 lacked proteinuria, and had no clinical evidence of HIVAN. The statistical analysis was performed blinded to the clinical diagnosis of both patients and controls.

For comparison of uNGAL expression in HIVAN with other types of kidney disease, cohorts of HIV-negative patients with AKI or CKD were used. Patients with AKI and non-biopsied stable CKD were derived from a previously described investigation of the ability of uNGAL to detect AKI in patients presenting to an emergency department (13). Patients with biopsy-proven glomerular or tubulointerstitial etiologies of CKD were also studied. These patients were >18-yr-old and underwent kidney biopsy as part of routine care. Urine was collected at the time of biopsy. Formalin-fixed tissues were embedded in paraffin, sectioned, and stained with hematoxylin/eosin, silver methenamine, and periodic acid-Schiff stain. Patients with AKI or CKD were stratified by type of kidney disease.

### Statistical Analysis

The program SAS 9.1 (Cary, N.C.) was used for statistical analyses. All continuous data were log-transformed before analyses and are presented as non-log-transformed values. Pearson's correlation was used to determine relationships among uNGAL and other continuous variables, and at test for unequal variances was used for comparisons. Fisher's exact test was used to compare categorical data among patients with and without HIVAN.

### Animals

TgFVB mice (17) were bred with FVB/N mouse strain to produce heterozygotes. The HIV transgene correlated with the presence of cataracts.

### Assays

The area of each kidney section was determined with Adobe Photoshop from 1× images, and total cysts per section were divided by this area to yield the cysts per unit area of each section.

NGAL was quantified by Western blots, using non-reducing 4 to 15% Tris-HCl gels Bio-Rad Laboratories, Inc., Hercules, Calif.) and monoclonal (1:1000; AntibodyShop, Gentofte, Denmark) or rabbit polyclonal antibodies (R&D Systems, Minneapolis, Minn.) together with standards (0.2 to 10.0 ng) of human or mouse recombinant NGAL protein. NGAL was reproducibly detected to 0.4 ng per lane. NGAL expression was quantified using ImageJ software (National Institute of Mental Health). NGAL mRNA was detected using digoxigenin-labeled antisense riboprobes generated from cDNAs encoding Ngal (exons 1 to 7, 566 bp) by linearization with XhoI followed by T7 RNA polymerase. Prussian blue staining in frozen sections used freshly prepared 2.5% potassium ferrocyanide and 2.5% hydrochloric acid for 20 min at room temperature.

Microarrays and real-time PCR utilized RNA isolated with the mirVANA RNA extraction kit (Ambion) and quantified by Nanoprop (Thermo Scientific, Delaware, U.S.) and gel electrophoresis. For real-time PCR analysis, samples were processed according to Bio-Rad SYBR GREEN, iCycler-MyiQ protocols. Target genes utilized respectively: Ngal 116 forward primer 5′-ctcagaact-tgatccctgcc-3′ (SEQ ID NO. 7) and NGALa 593 reverse 5′-tccttgaggcccagacactt-3′ (SEQ ID NO. 8); β-actin 415 forward primer 5′-ctaaggccaaccgtgaaaag-3′ (SEQ ID NO. 9) and 13-actin 696 reverse primer 5′-tctcagctgtggtggtgaag-3′ (SEQ ID NO. 10). The ΔΔCT method was used to calculated fold amplification of transcripts. For microarray analysis, double-stranded cDNA was synthesized from total RNA (7 μg) extracted from whole kidneys, and in vitro transcription biotin-labeled cRNA was generated for GeneChip hybridization (GeneChip One-Cycle Target Labeling Kit, Affymetrix). Fragmented biotin-labeled cRNA was hybridized to Affymetrix Mouse Genome 430 2.0 Gene-Chips. The results of the Affymetrix chip experiments were normalized using GC-RMA (Bioinformatics Toolbox, Matlab R2008a, The Mathworks, Inc.). After standardization using WT information, the data were averaged. Given the distribution of expression profiles, the Bonferroni-corrected significance threshold was calculated to 3.17×10⁻⁶.

### References for Example 2

1. UNAIDS: Report on the Global AIDS Epidemic, 2008. Available at http://www.unaids.org/en/knowledgecentre/hivdata/globalreport/2008/defaultasp. Accessed May 2009
2. Rao T K, Filippone E J, Nicastri A D, Landesman S H, Frank E, Chen C K, Friedman E A: Associated focal and segmental glomerulosclerosis in the acquired immunodeficiency syndrome. N Engl J Med 310: 669-673, 1984
3. Winston J A, Klotman M E, Klotman P E: HIV-associated nephropathy is a late, not early, manifestation of HIV-1 infection. Kidney Int 55: 1036-1040, 1999
4, Lucas G M, Eustace J A, Sozio S, Mentari E K, Appiah K A, Moore R D: Highly active antiretroviral therapy and the incidence of HIV-1-associated nephropathy: A 12-year cohort study. AIDS 18: 541-546, 2004
5. Shahinian V, Rajaraman S, Borucki M, Grady J, Hollander W M, Ahuja T S: Prevalence of HIV-associated nephropathy in autopsies of HIV-infected patients. Am J Kidney Dis 35: 884-888, 2000
6. Atta M G, Lucas G M, Fine D M: HIV-associated nephropathy: Epidemiology, patho-genesis, diagnosis and management. Expert RevAnti Infect Ther 6: 365-371, 2008
7. Wyatt C M, Klotman P E, D'Agati V D: HIV-associated nephropathy: Clinical presentation, pathology, and epidemiology in the era of antiretroviral therapy. Semin Nephrol 28: 513-522, 2008
8. Herman E S, Klotman P E: HIV-associated nephropathy: Epidemiology, pathogenesis, and treatment. Semin Nephrol 23: 200-208, 2003
9. Bruggeman L A, Dikman S, Meng C, Quaggin S E, Coffman T M, Klotman P E: Nephropathy in human immunodeficiency virus-1 transgenic mice is due to renal transgene expression. J Clin Invest 100: 84-92,1997
10. Post F A, Campbell L J, Hamzah L, Collins L, Jones R, Siwani R, Johnson L, Fisher M, Holt S G, Bhagani S, Frankel A H, Wilkins E, Ainsworth J G, Larbalestier N, Macallan D C, Banerjee D, Baily G, Thuraisingham R C, Donohoe P, Hendry B M, Hilton R M, Edwards S G, Hangartner R, Howie A J, Connolly J O, Easterbrook P J: Predictors of renal outcome in HIV-associated nephropathy. Clin Infect Dis 46: 1282-1289, 2008
11, Gupta S K, Eustace J A, Winston J A, Boydstun I I, Ahuja T S, Rodriguez R A, Tashima K T, Roland M, Franceschini N, Patella F J, Lennox J L, Klotman P E, Nachman S A, Hall S D, Szczech L A: Guidelines for the management of chronic kidney disease in HIV-infected patients: Recommendations of the HIV Medicine Association of the Infectious Diseases Society of America. Clin Infect Dis 40: 1559-1585,2005
12. Szczech L A, Gupta S K, Habash R, Guasch A, Kalayjian R, Appel R, Fields T A, Svetkey L P, Flanagan K H, Klotman P E, Winston J A: The clinical epidemiology and course of the spectrum of renal diseases associated with HIV infection. Kidney Int 66: 1145-1152, 2004
13. Nickolas T L, O'Rourke M J, Yang J, Sise M E, Canetta P A, Barasch N, Buchen C, Khan F, Mori K. Giglio J, Devarajan P, Barasch J: Sensitivity and specificity of a single emergency department measurement of urinary neutrophil gelatinase-associated lipocalin for diagnosing acute kidney injury. Ann Intern Med 148: 810-819, 2008
14. Mori K, Lee H T, Rapoport D, Drexler I R, Foster K, Yang J, Schmidt-Ott K M, Chen X, Li J Y, Weiss S. Mishra J, Cheema F H, Markowitz G, Suganami T, Sawai K, Mukoyama M, Kunis C, D'Agati. V, Devarajan P, Barasch J: Endocytic delivery of lipocalin—siderophore-iron complex rescues the kidney from ischemia-reperfusion injury. J Clin Invest 115: 610-621, 2005
15. Mishra J, Ma Q, Prada A, Mitsnefes M, Zahedi K, Yang J, Barasch J, Devarajan P: Identification of neutrophil gelatinase-associated lipocalin as a novel early urinary biomarker for ischemic renal injury, J Am Soc Nephrol 14: 2534-2543, 2003
16, Schmidt-Ott K M, Mori K, Li J Y, Kalandadze A, Cohen D J, Devarajan P. Barasch J: Dual action of neutrophil gelatinase-associated lipocalin. J Am Soc Nephrol 18: 407-413, 2007
17. Kopp J B, Klotman M E, Adler S H, Bruggeman L A, Dickie P, Marinos N J, Eckhaus M, Bryant J L, Notkins A L, Klotman P E: Progressive glomerulosclerosis and enhanced renal accumulation of basement membrane components in mice transgenic for human immunodeficiency virus type 1 genes. Proc Natl Acad Sci USA 89: 1577-1581, 1992
18. Kaufmann R, Laroche D, Buchner K, Hucho F, Rudd C, Lindschau C, Ludwig P, Hoer A, Oberdisse E, Kopp J, Korner I J, Repke H: The HIV-1 surface protein gp1.20 has no effect on transmembrane signal transduction in T cells. J Acquir Immune Defic Syndr 5: 760-770, 1992
19. Vaidya V S, Ferguson M A, Bonventre J V: Biomarkers of acute kidney injury. Annu Rev Pharmacol Toxicol 48: 463-493, 2008
20. Nankivell B J, Boadle R A, Harris D C: Iron accumulation in human chronic renal disease. Am J Kidney Dis 20: 580-584, 1992
21. Bolignano D, Coppolino G, Campo S, Aloisi C, Nicocia G, Frisina N, Buemi M: Neutrophil gelatinase-associated lipocalin in patients with autosomal-dominant polycystic kidney disease. Am J Nephrol 27: 373-378, 2007
22. Yang J, Goetz D, Li J Y, Wang W, Mori K, Setlik D, Du T, Erdjument-Bromage H, Tempst P. Strong R, Barasch J: An iron delivery pathway mediated by a lipocalin. Mol. Cell 10: 1045-1056, 2002
23, Gwira J A, Wei F, Ishibe S. Ueland J M, Barasch J. Cantley L G: Expression of neutrophil gelatinase-associated lipocalin regulates epithelial morphogenesis in vitro. J Biol Chem 280: 7875-7882, 2005
24. Iannetti A, Pacifico F, Acquaviva R, Lavorgna A, Crescenzi E, Vascotto C, Tell G, Salzano A M, Scaloni A, Vuttariello E, Chiappetta G, Formisano S, Leonardi A: The neutrophil gelatinase-associated lipocalin (NGAL), a NF-B-regulated gene, is a survival factor for thyroid neoplastic cells. Proc Natl Acad Sci USA 105: 14058-14063, 2008
25. Playford R J, Belo A, Poulsom R, Fitzgerald A J, Harris K, Pawluczyk I, Ryon J, Darby T, Nilsen-Hamilton M, Ghosh S. Marchbank T: Effects of mouse and human lipocalin homologues 24p3/lcn2 and neutrophil gelatinase-associated lipocalin on gastrointestinal mucosal integrity and repair. Gastroenterology 131: 809-817, 2006
26. Wei F, Karihaloo A, Yu Z, Marlier A, Seth P. Shibazaki S, Wang T, Sukhatme V P, Somlo S, Cantley L G: Neutrophil gelatinase-associated lipocalin suppresses cyst growth by Pkd1 null cells in vitro and in vivo, Kidney Int 74: 1310-1318, 2008
27. Ray P E, Liu X H, Robinson L R, Reid W, Xu L, Owens J W, Jones O D, Denaro F, Davis H G, Bryant J L: A novel HIV-1 transgenic rat model of childhood HIV-1-associated nephropathy. Kidney Int 63: 2242-2253, 2003
28. Wyatt C M, Klotman P E: HIV-1 and HIV-associated nephropathy 25 years later. Clin Am Soc Nephrol 2: S20-S24, 2007
29. Wyatt C M, Morgello S, Katz-Malamed R, Wei C, Klotman M E, Klotman P E, D'Agati, V D: The spectrum of kidney disease in patients with AIDS in the era of antiretroviral therapy. Kidney Int 75: 428-434, 2009
30. Levey A S, Bosch J P, Lewis J B, Greene T, Rogers N, Roth D: A more accurate method to estimate glomerular filtration rate from serum creatinine: A new prediction equation. Modification of Diet in Renal Disease Study Group. Ann Intern Med 130: 461-470, 1999.

### Example 3

### uNGAL and uL-FABP in HIVAN

We performed a comparative study of diverse proteinuric glomerulopathies in 25 patients who were HIV-positive. 18 patients had HIVAN and 7 had other glomerulopathies (4 MPGN, 1 MGN, 1 amyloid and 1 malarial GN). HIVAN and non-HIVAN patients did not differ with respect to age, ethnicity, serum creatinine, eGFR, proteinuria or the prevalence of hypocomplementemia (6% v 29%, p=0.18), but HIVAN patients were less likely to have HCV infections. HIVAN patients expressed 4 fold higher levels of uNGAL than other glomerulopathies (387±338 v 94±101 μg/g uCr, p=0.02). At a cutpoint of 121.5 μg uNGAL/g uCr demonstrated 94% sensitivity and 71% specificity for the diagnosis of HIVAN, with an area under the ROC curve of 0.88. Thus, uNGAL can distinguish HIVAN from other proteinuric glomerulopathies in the HIV-infected patient, likely because of its specific expression from characteristic microcysts

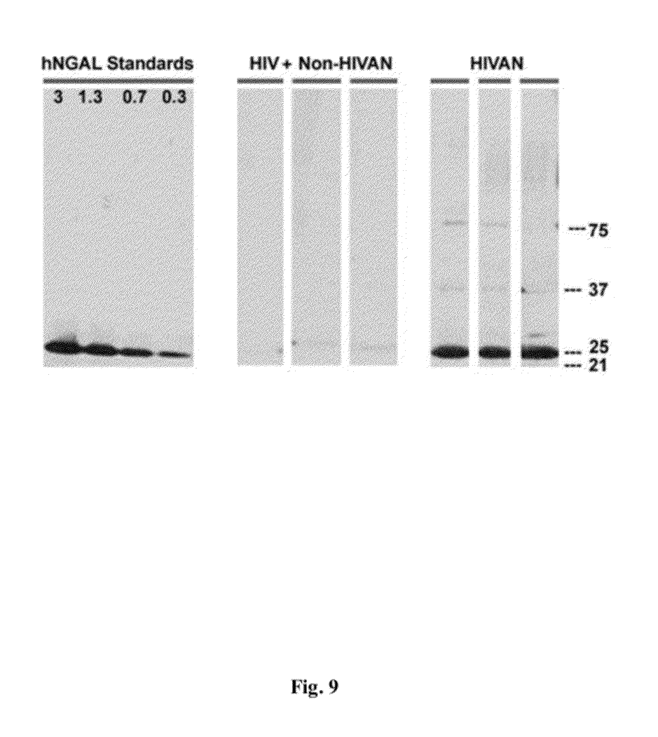

Twenty five HIV positive patients underwent renal biopsy and donated urine at the time of the biopsy. They were compared with age-, race-, and gender-matched HIV-negative control subjects. Renal biopsies were processed by light microscopy, immunofluorescence and electron microscopy Urine samples were stored at −80° C. until uNGAL (10 μL) was quantified by immunoblots with non-reducing 4% to 15% gradient polyacrylamide gels (Bio-Rad Laboratories) using standards (0.3-3 ng) of human recombinant NGAL protein (FIG. 9). Immunoblots were used to detect and quantify the monomeric form of NGAL; immunoblotting is necessary since some CKD patients express complexes of NGAL linked to other proteins, whereas only the monomeric form has been validated epidemiologically in association with renal disease (Nickolas and Barasch, unpublished data). Urinary L-FABP (uL-FABP) was measured using a sandwich-type ELISA kit (CMIC Co., Ltd). Urinary NAG (uNAG) was measured using a colorimetric assay kit (Roche Applied Sciences). Investigators were blinded while performing the assays.

The data are represented both as concentration and as the absolute values corrected for urinary creatinine Both measurements are reported for the following reason: a normalized ratio can be misleading in cases of acute renal failure because of a rapid decline in creatinine excretion which would amplify the urinary biomarker signal (Waikar, S S, Sabbisetti, V S, Bonventre, T V: Normalization of urinary biomarkers to creatinine during changes in glomerular filtration rate. Kidney Int, 78: 486-494, 2010.). However, in patients with chronic kidney disease, the rate change of GFR is much slower such that production and excretion of creatinine approximate one another until end stage renal disease is reached. Hence, we report both the absolute biomarker level normalized to urinary creatinine as well as its urinary concentration. Statistical analysis was performed with Stata 10.1.

The cohort consisted of 18 patients with HIVAN, 4 patients with membranoproliferative glomerulonephritis (MPGN), 1 with membranous glomerulopathy, 1 with malarial glomerulonephritis, and 1 with renal AA amyloidosis. Baseline characteristics of our cohort are presented in Table 3.

Patients with HIVAN and non-HIVAN glomerulopathies were comparable with respect to age, renal function (BUN, sCr and eGFR), nutritional status (serum albumin and cholesterol), proteinuria and complement levels. In addition, CD4 counts, HCV co-infection, and use of highly active antiretroviral therapy did not differ between the groups. (Table 3).

Compared to HIV-positive patients with non-HIVAN glomerulopathies, patients with HIVAN had significantly elevated levels of uNGAL as well as urinary liver fatty acid binding protein (uL-FABP), a second indicator of tubular injury. However, there was no difference in urinary N-acetyl-β-d-glucosaminidase (uNAG) levels between the two groups of patients (Table 1). We also found significantly higher levels of uNGAL and uL-FABP in HIVAN patients compared to our HIV-negative controls.

A significant correlation between uNGAL (μg/g) and uL-FABP (μg/g) (r=0.52, p=0.007) but not between these two biomarkers and uNAG (μg/g) (r=0.34 and r=0.17 respectively, p=0.1 and p=0.42) was present in our cohort of 25 HIV positive patients. Neither uNGAL nor uL-FABP correlated with sCr, eGFR (MDRD) BUN, proteinuria (for uNGAL: r=0.31, p=0.12; r=0.131 p=0.603; r=−0.02, p=0.92; r=0.23, p=0.33; for uL-FABP r=0.09, p=0.67; r=0.241, p=0.335; r=0.14, p=0.56; r=−0.08, p=0.75; respectively). Urinary NGAL levels were not influenced by the use of HAART (Not on HAART 246±285 vs. On HAART 407±309, p=0.45), hypocomplementemia (108±98 vs. 297±322, p=0.28) or HCV infection (159±128 vs. 236±312, p=0.65) and did not show any correlation with CD4 counts (−0.004, p=0.99). Among HIVAN patients the percentage of collapsed glomeruli correlated with the sCr and 24-hour proteinuria (r=0.49, p=0.01; r=0.48, p=0.03, respectively) but not with either uNGAL (r=0.29, p=0.15) or with uL-FABP (r=0.1, p=0.64).

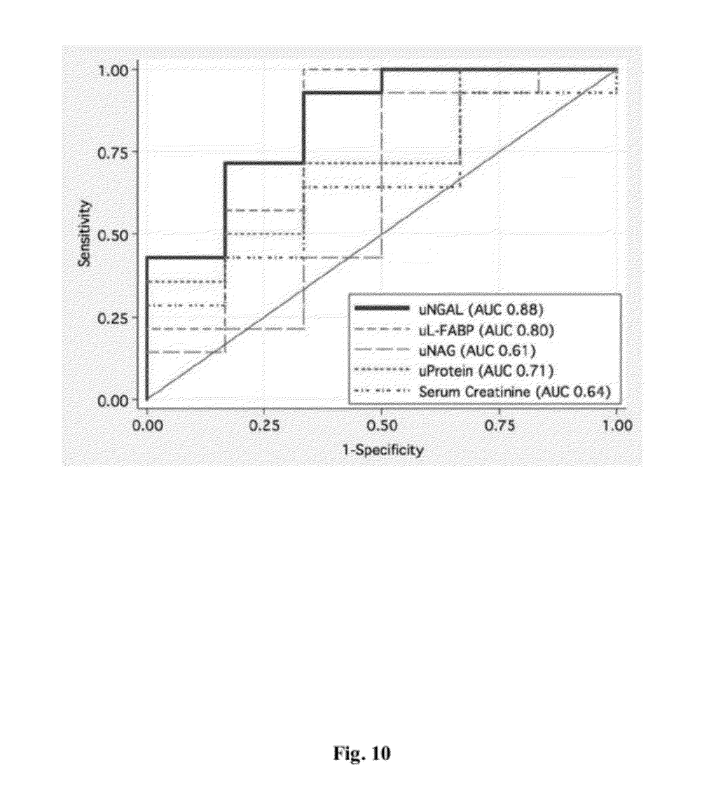

The ROC curve for uNGAL (μg/g of uCr) had an AUC of 0.88 (FIG. 10). The concentration of uNGAL with the highest Youden index was 121.5 μg/g uCr, which had a sensitivity of 94% and specificity of 71% (Table 4) for detection of HIVAN. At a cutpoint of 121.5 μg/g uCr, 88% of cases were correctly classified with a positive likelihood ratio of 3.3 and a positive predictive value of 89%. The ROC curve for uL-FABP (μg/g uCr) had an AUC of 0.80, and the threshold of 116 μg/g uCr had similar sensitivity and specificity (χ2=1.74, p=0.2; FIG. 10 and Table 4). By contrast, the discriminatory ability to distinguish HIVAN from other glomerulopathies was significantly worse for uNAG and the more traditional markers such as sCr and proteinuria (FIG. 10 and Table 5).

Renal epithelium is a distinct compartment for HIV-1 infection and replication. The kidney responds by glomerular collapse, microcystic tubular dilatation and epithelial simplification. In a mouse model of HIVAN, NGAL mRNA was 100-fold higher than in littermate controls, due to its specific expression by medullary tubules that had undergone microcystic dilation. Consistently, uNGAL was present in HIV-positive children and adults with HIVAN. Thus uNGAL appears to be robustly expressed in HIVAN more so than other forms of CKD.

The ROC curve for uNGAL indicated excellent diagnostic utility to detect HIVAN (FIG. 10). Similarly, while the ROC curve was not as impressive for uL-FABP, it was not significantly different from uNGAL. Both markers are expressed by the tubular portion of the nephron upon HIV-cystic in the collecting ducts or TALH(NGAL) or upon tubulointerstitial damage of the proximal nephron (uL-FABP). These data stand in contrast to measures of glomerular function, such as eGFR and proteinuria which were less useful predictors of HIVAN (FIG. 9; Table 5). Proteinuria vs eGFR and proteinuria vs collapsed glomeruli were both correlated but neither proteinuria nor eGFR could distinguish HIVAN from other forms of proteinuric renal disease. Conversely, uNGAL concentrations failed to correlate with GFR. In sum, it appears that two markers of tubular damage more specifically detect disease progression than markers of glomerular filtration, but we do not have an explanation for the failure of uNAG, which is released following structural damage to the tubular cells, including subclinical injury related to tenofovir, predict HIVAN.

We conclude that HIVAN can be identified by 2 genes expressed in response to tubular rather than glomerular damage. uNGAL provided the best non-invasive clinical marker of HIVAN. Our data suggest that uNGAL will be useful in parts of the world where HIVAN is prevalent but renal biopsy is not practical, and that uNGAL predicts progression in cystic-HIV CKD but not in other forms of CKD.

TABLE 3
Baseline characteristics of the cohort

| | HIV negative | | | |
|---|---|---|---|---|
| Characteristic | controls | Non - HIVAN | HIVAN | p value* |
| N | 24 | 7 | 18 | |
| Age (yrs) | 43.7 ± 11.1 | 44.8 ± 12.3 | 43.5 ± 10.4 | 0.8 |
| Black (%) | 88 | 86 | 89 | 0.8 |
| BUN (mg/dL) | 11.1 ± 3.8 | 40.5 ± 25.7 | 43.6 ± 24.2 | 0.8 |
| Creatinine (mg/dL) | 0.9 ± 0.2 | 2.8 ± 1.6 | 4.1 ± 2.6 | 0.2 |
| eGFR (MDRD) mL/min | 110.8 ± 18 | 46.6 ± 35.8 | 31.5 ± 26.7 | 0.3 |
| Urine protein (g/24 hrs) | N/A | 7.9 ± 8 | 9.4 ± 7.8 | 0.7 |
| Urine protein (g/g of creatinine) | N/A | 5.5 ± 4.1 | 10.6 ± 7.7 | 0.14 |
| Serum albumin (g/dL) | 4.1 ± 0.5 | 2.5 ± 0.9 | 2.4 ± 0.8 | 0.7 |
| Serum Cholesterol (mg/dL) | N/A | 218 ± 99 | 211 ± 99 | 0.9 |
| Urinary NGAL (ng/mL) | 20.1 ± 21.0 | 38.6 ± 22.7 | 191.7 ± 186.9 | 0.04 |
| Urinary NGAL (μg/g of | 30.6 ± 39.2 | 94 ± 100.7 | 387.2 ± 339.7 | 0.04 |
| creatinine) | | | | |
| Urinary L-FABP (ng/mL) | 13.9 ± 27.8 | 59.2 ± 27.3 | 169.5 ± 105.5 | 0.01 |
| Urinary L-FABP (μg/g of | 14.8 ± 27.7 | 148.9 ± 153.2 | 343.0 ± 186.5 | 0.02 |
| creatinine) | | | | |
| Urinary NAG (mmol/L) | N/A | 5.18 ± 4.19 | 7.1 ± 8.6 | 0.59 |
| Urinary NAG (mmol/g of | N/A | 9 ± 7 | 12 ± 8 | 0.41 |
| creatinine) | | | | |

*p values are for a comparison of HIV patients with and without HIVAN.

TABLE 4
Urine NGAL and urine LFABP test ROC curve characteristics.

| | uNGAL (μg/g) | | | | | uL-FABP (μg/g) | | | |
|---|---|---|---|---|---|---|---|---|---|
| | 73.8 | 121.5 | 144.4 | 182 | 320 | 55.8 | 116.2 | 312.5 | 589.8 |
| Sensitivity (%) | 100 | 94.4 | 77.8 | 66.7 | 50 | 100 | 94.4 | 55.6 | 22 |
| Specificity (%) | 57.1 | 71.4 | 85.7 | 85.7 | 86 | 28.6 | 71.4 | 85.7 | 100 |
| Youden Index | 0.57 | 0.66 | 0.63 | 0.52 | 0.44 | 0.29 | 0.66 | 0.41 | 0.22 |
| Correctly Classified (%) | 88 | 88 | 80 | 72 | 60 | 80 | 88 | 64 | 44 |
| Positive Likelihood | 2.3 | 3.3 | 5.4 | 4.7 | 3.6 | 1.4 | 3.3 | 3.9 | — |
| Ratio | | | | | | | | | |
| Negative Likelihood | — | 0.08 | 0.3 | 0.4 | 0.6 | 0 | 0.08 | 0.5 | 0.78 |
| Ratio | | | | | | | | | |
| Positive Predictive | 86 | 89 | 93 | 92 | 90 | 78 | 89 | 91 | 100 |
| Value | | | | | | | | | |
| Negative Predictive | 100 | 82 | 0.60 | 50 | 0.40 | 0.22 | 82 | 9.43 | 0.33 |
| Value | | | | | | | | | |

TABLE 5
Comparison of the ROC curves for different
markers of renal injury.

| Non-invasive marker | AUC | 95% Confidence Interval |
|---|---|---|
| Urinary NGAL (μg/g of creatinine) | 0.88 | 0.71-1.00 |
| Urinary LFABP (μg/g of creatinine) | 0.80 | 0.57-1.00 |
| Urinary NAG (mmol/g of creatinine) | 0.61 | 0.33-0.90 |
| Urinary Protein (g/24 hrs) | 0.71 | 0.46-0.97 |
| Serum Creatinine (mg/dL) | 0.64 | 0.37-0.91 |

χ² = 14.57, p = 0.0057

Although embodiments of the invention have been described and illustrated in the foregoing, it is understood that the present disclosure has been made only by way of example, and that numerous changes in the details of implementation can be made without departing from the spirit and scope of the invention. Features of the disclosed embodiments can be combined and rearranged in various ways within the scope and spirit of the invention.

What is claimed is:

1. A method for diagnosing HIV associated nephropathy (HIVAN) in a subject, the method comprising determining the amount of NGAL protein present in a sample of urine from a subject, wherein an amount of NGAL protein present in the sample that exceeds about 100 μg/g creatinine indicates that the subject may have HIVAN.

2. The method of claim 1, wherein an amount of NGAL protein present in the sample that exceeds about 110 μg/g creatinine indicates that the subject may have HIVAN.

3. The method of claim 1, wherein an amount of NGAL protein present in the sample that exceeds about 115 μg/g creatinine indicates that the subject may have HIVAN.

4. The method of claim 1, wherein an amount of NGAL protein present in the sample that exceeds about 120 μg/g creatinine indicates that the subject may have HIVAN.

5. The method of claim 1, wherein the subject is of African descent.

6. The method of claim 1, wherein the subject has already been determined to be HIV positive.

7. The method of claim 1, wherein the subject has no signs of kidney disease.

8. The method of claim 1, further comprising subsequently treating the HIVAN in the subject.

9. The method of claim 1, further comprising subsequently treating the subject with highly active antiretroviral therapy (HAART).

10. The method of claim 1, wherein an amount of NGAL protein measured that exceeds about 100 μg/g creatinine indicates that the subject may have microcystic degeneration of the kidney.

11. The method of claim 1, wherein an amount of NGAL protein present in the sample that exceeds about 100 μg/g creatinine indicates that the subject may have collapsing focal segmental glomerulosclerosis.

12. The method of claim 1, further comprising determining the amount of uL-FABP protein present in a sample of urine from the subject, wherein an amount of uL-FABP protein present in the sample that exceeds about 100 μg/g creatinine indicates that the subject may have HIVAN.

13. The method of claim 12, wherein an amount of uL-FABP protein present in the sample that exceeds about 110 μg/g creatinine indicates that the subject may have HIVAN.

14. The method of claim 12, wherein an amount of uL-FABP protein present in the sample that exceeds about 115 μg/g creatinine indicates that the subject may have HIVAN.

15. The method of claim 1, further comprising determining the amount of KIM-1 protein present in a sample of urine from the subject, wherein an amount of NGAL protein that is more than about 8-fold higher than the amount of KIM-1 protein, in combination with an amount of NGAL protein that exceeds about 100 μg/g creatinine, indicates that the subject may have HIVAN.

16. The method of claim 1, further comprising determining whether the subject has proteinuria, wherein proteinuria, in combination with an amount of NGAL protein that exceeds about 100 μg/g creatinine, indicates that the subject may have HIVAN.

17. The method of claim 1, further comprising determining the subject's CD4 count, wherein a CD4 count of less than 200 cells/mm³, in combination with an amount of NGAL protein that exceeds about 100 μg/g creatinine, indicates that the subject may have HIVAN.

18. The method of claim 1, further comprising measuring the subject's serum creatinine level, wherein a serum creatinine level of more than about 1.5 mg/dl, in combination with an amount of NGAL protein that exceeds about 100 μg/g creatinine, indicates that the subject may have HIVAN.

19. The method of claim 1, further comprising determining the subject's HIV viral load, wherein an HIV viral load of more than about 100,000 IU/ml, in combination with an amount of NGAL protein that exceeds about 100 μg/g creatinine, indicates that the subject may have HIVAN.

20. The method of claim 1, wherein the NGAL protein is monomeric NGAL.

21. The method of claim 1, wherein the step of measuring NGAL protein comprises contacting the sample with an antibody that binds to the NGAL protein.

22. The method of claim 21, comprising performing an enzyme-linked immunosorbent assay (ELISA).

23. The method of claim 21, wherein the antibody is immobilized on a solid support.

24. The method of claim 23, wherein the solid support comprises a dipstick or a test strip.

25. The method of claim 21, comprising performing an immunoblotting method.

26. The method of claim 21, comprising performing an immunoblotting method to detect monomeric NGAL.


SEQUENCE LISTING (10 sequences)

SEQ ID NO: 1 - DNA, 20 bp, Artificial Sequence
  Description of Artificial Sequence Synthetic primer
ctcagaacttgatccctgcc

SEQ ID NO: 2 - DNA, 20 bp, Artificial Sequence
  Description of Artificial Sequence Synthetic primer
tccttgaggcccagacactt

SEQ ID NO: 3 - DNA, 25 bp, Artificial Sequence
  Description of Artificial Sequence Synthetic primer
ccaggcactgtggattcttatgtgg

SEQ ID NO: 4 - DNA, 25 bp, Artificial Sequence
  Description of Artificial Sequence Synthetic primer
tgtgacatcctcaggagatacctgg

SEQ ID NO: 5 - DNA, 20 bp, Artificial Sequence
  Description of Artificial Sequence Synthetic primer
ctaaggccaaccgtgaaaag

SEQ ID NO: 6 - DNA, 20 bp, Artificial Sequence
  Description of Artificial Sequence Synthetic primer
tctcagctgtggtggtgaag

SEQ ID NO: 7 - DNA, 20 bp, Artificial Sequence
  Description of Artificial Sequence Synthetic primer
ctcagaacttgatccctgcc

SEQ ID NO: 8 - DNA, 20 bp, Artificial Sequence
  Description of Artificial Sequence Synthetic primer
tccttgaggcccagacactt

SEQ ID NO: 9 - DNA, 20 bp, Artificial Sequence
  Description of Artificial Sequence Synthetic primer
ctaaggccaaccgtgaaaag

SEQ ID NO: 10 - DNA, 20 bp, Artificial Sequence
  Description of Artificial Sequence Synthetic primer
tctcagctgtggtggtgaag